(19) United States
(12) Patent Application Publication
(10) Pub. No.: US 2002/0137199 A1
(43) Pub. Date: Sep. 26, 2002

(54) Micro storage, reaction and detection cells and method and apparatus for use thereof

(76) Inventors: Michael J. Jobin, Boston, MA (US); Andrew R. Muir, Cohasset, MA (US); David M. Sykes, Cambridge, MA (US); James L. Woodward, Milton, MA (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 10/011,237

(22) Filed: Oct. 22, 2001

Related U.S. Application Data
(60) Provisional application No. 60/243,369, filed on Oct. 27, 2000.
(60) Provisional application No. 60/290,225, filed on May 11, 2001.
(60) Provisional application No. 60/307,122, filed on Jul. 23, 2001.

(51) Int. Cl. C12M 1/34

(52) U.S. Cl. 435/287.2

(57) ABSTRACT

Disclosed are devices and methods for the storage, shipment, moving and/or transferring, mixing, reacting, isolation, conditioning, lyophilization, stabilization, metering, dispensing, detecting, manipulation and management of materials, particularly solids, powders and liquids. Each such device or method provides a means of storing one or more quantities of one or more materials in one or more reservoirs, optionally protecting said quantities from air, moisture or light. Optionally, multiple such reservoirs may be placed or arranged in ensembles such as in arrays or other physical patterns, optionally providing for the simultaneous, parallel or rapid access to more than one such reservoir at once. Devices and methods of the invention may also be configured to deliver material from a reservoir in such manner that a defined or desired quantity of material is dispensed from the reservoir or that such dispensing is performed at a defined or desired rate or that the material is dispensed until one or more criteria are met for such dispensing to cease.

### CROSS REFERENCE TO RELATED APPLICATIONS

This application claims the priority of Jobin, Sykes, Woodward and Muir, U.S. Provisional Application No. 60/243,369 filed Oct. 27, 2000 entitled, DEVICES AND METHODS FOR MATERIAL STORAGE AND DISPENSING; of Jobin, Muir and Woodward, U.S. Provisional Application No. ______ filed Apr. 28, 2001 entitled, NOVEL MICRO STORAGE REACTION AND DETECTION CELLS WITH APPARATUS FOR MANIPULATION AND METHODS OF USE; and of Jobin and Muir, U.S. Provisional Application No. ______ filed Jul. 19, 2001 entitled, NOVEL MICRO REACTION AND DETECTION DEVICES AND APPARATUS AND METHODS OF USE, the whole of which are hereby incorporated by reference herein.

### STATEMENT REGARDING FEDERALLY SPONSORED RESEARCH OR DEVELOPMENT

NONE

### BACKGROUND OF THE INVENTION

The field of pharmaceutical research is undergoing rapid changes in the drive to develop new treatments for disease. Driven by the pressure to produce more leads or potential drugs, researchers are performing large numbers of tests to screen whether a compound is suitable for further development, a process known as High Throughput Screening (HTS). In addition, researchers are testing large quantities of genetic material and derived proteins in an effort to better understand the mechanisms of disease.

As the number of tests performed has increased several orders of magnitude in the past decade, assay processes have become highly automated, requiring large investments in capital equipment such as robotics. Existing techniques may waste much of the expensive reagents and compounds that are ultimately tested. The great expense of the process has limited the number of researchers able to participate. Thus, devices and methods are needed for the reliable and controlled manipulation of very small quantities of material in a commercially advantageous manner.

### BRIEF SUMMARY OF THE INVENTION

The invention is directed to devices and methods for the storage, shipment, moving and/or transferring, mixing, reacting, isolation, conditioning, lyophilization, stabilization, metering, dispensing, detecting, manipulation and management of materials, particularly solids, powders and liquids. Each such device or method provides a means of storing one or more quantities of one or more materials in one or more reservoirs, optionally protecting said quantities from air, moisture or light. Optionally, multiple such reservoirs may be placed or arranged in ensembles such as in arrays or other physical patterns, optionally providing for the simultaneous, parallel or rapid access to more than one such reservoir at once. Devices and methods of the invention may also be configured to deliver material from a reservoir in such manner that a defined or desired quantity of material is dispensed from the reservoir or that such dispensing is performed at a defined or desired rate or that the material is dispensed until one or more criteria are met for such dispensing to cease.

In preferred embodiments, the invention is directed to microscopic storage cells containing samples of liquids, solids, powders or other forms of matter or combinations of same and to the use thereof. Such cells are of dimensions such that they may hold a volume of sample defined by the volume of the cell, or the sample volume may be less than the cell volume. Said volumes may advantageously be on the order of one microliter for many commercially useful applications, but greater or lesser volumes may be featured also and are all covered by the present invention. Most preferably, a liquid sample is drawn into and retained in a cell by capillary action.

Devices taught by the invention are, in one preferred embodiment, formed as thin laminar sheets of chemically inert, e.g., plastic material containing multiple storage cells advantageously laid out to match standard 96, 384 or 1536 well plates. Sample material is inserted into such cells, which are optionally sealed, then optionally stored. Liquid, optionally including reagents, may subsequently be added and the stored material dissolved and mixed, and chemical reactions induced. The reagents and/or chemical reactions and/or chemical products may be detected. Detection and qualitative or quantitative analysis may be conducted such as by optical, electrochemical or other known means. Apparatus for manipulating said plastic sheets and the storage cells therein also is covered by this invention.

The devices and methods of the invention provide for the efficient manipulation of materials and low loss and/or wastage, with consequent cost reductions. The low cost of the devices according to the invention and the efficient utilization of materials therein may make disposability an attractive or preferred option. Ensembles of devices according to the invention may be implemented in such manner that automation or robotic usage is facilitated, with additional benefits resulting from overall efficient usage. Thus, these devices and methods are of particular commercial utility for the efficient manipulation of chemical reagents such as those used in combinatorial chemistry, high throughput screening of drug candidates or for such other applicable uses as will be apparent to those skilled in the art.

### BRIEF DESCRIPTION OF THE SEVERAL VIEWS OF THE DRAWING

Other features and advantages of the invention will be apparent from the following description of the preferred embodiments thereof and from the claims, taken in conjunction with the accompanying drawings, in which:

FIGS. 1a-1h show plan and section views of devices and methods of use according to the invention;

FIGS. 2a-2f show section views of devices and methods of use according to the invention;

FIGS. 3a-3d show section views of devices and methods of use according to the invention;

FIGS. 4a-4l show section views of alternative embodiments of devices and methods of use according to the invention;

FIGS. 5a-5e show section views of alternative embodiments of devices and methods of use according to the invention;

FIGS. 6a-6b show section views of devices and methods of use according to the invention;

FIGS. 7a-7b show section views of devices and methods of use according to the invention;

FIGS. 8a-8b show plan views of devices and methods of use according to the invention;

FIG. 9 show plan and section views of alternative embodiments of devices and methods of use according to the invention;

FIG. 10 show a plan view of an alternative embodiment of devices and methods of use according to the invention;

FIG. 11 show a plan view of an alternative embodiment of devices and methods of use according to the invention;

FIG. 12 shows a view of a device and method of use according to the invention;

FIG. 13 shows a section view of a device and method of use according to the invention;

FIG. 14 shows a section view of an alternative embodiment of a device and method of use according to the invention;

FIG. 15 shows a section view of an alternative embodiment of a device and method of use according to the invention;

FIG. 16 shows a section view of a device and method of use according to the invention;

FIG. 17 shows a section view of an alternative embodiment of a device and method of use according to the invention;

FIGS. 18a-18i show section views of devices and methods of use according to the invention;

FIGS. 19a-19c show section views an alternative embodiment of devices and methods of use according to the invention;

FIG. 20 shows a section view of an alternative embodiment of devices and methods of use according to the invention;

FIG. 21 shows section views an alternative embodiment of devices and methods of use according to the invention;

FIGS. 22a-22c show section views of alternative embodiments of devices and methods of use according to the invention;

FIG. 23 shows section views of an alternative embodiment of devices and methods of use according to the invention;

FIG. 24 show section views of an alternative embodiment of devices and methods of use according to the invention;

FIG. 25 shows an isometric view of an alternative embodiment of devices and methods of use according to the invention;

FIG. 26 shows an isometric view of an alternative embodiment of devices and methods of use according to the invention;

FIG. 27 shows a section view of an alternative embodiment of devices and methods of use according to the invention;

FIG. 28 shows a section view of an alternative embodiment of devices and methods of use according to the invention;

FIGS. 29a-29c show section views of alternative embodiments of devices and method of use according to the invention;

FIG. 30 shows a section view of an alternative embodiment of devices and methods of use according to the invention;

FIG. 31 shows a section view of an alternative embodiment of devices and methods of use according to the invention;

FIG. 32 shows a section view of an alternative embodiment of devices and methods of use according to the invention;

FIG. 33 shows a section view of an alternative embodiment of devices and methods of use according to the invention;

FIG. 34 shows a section view of an alternative embodiment of devices and methods of use according to the invention;

FIG. 35 shows a section view of an alternative embodiment of devices and methods of use according to the invention;

FIG. 36 shows a section view of an alternative embodiment of devices and methods of use according to the invention;

FIGS. 37a-37b show section and isometric views of an alternative embodiment of devices and methods of use according to the invention;

FIG. 38 shows a section view of an alternative embodiment of devices and methods of use according to the invention;

FIGS. 39a-39d show section views of an alternative embodiment of devices and methods of use according to the invention;

FIGS. 40a-40d show section views of an alternative embodiment of devices and methods of use according to the invention;

FIG. 41 show a section view of an alternative embodiment of devices and methods of use according to the invention;

FIGS. 42a-42c show section views of an alternative embodiment of devices and methods of use according to the invention;

FIGS. 43a-43d show section views of an alternative embodiment of devices and methods of use according to the invention;

FIGS. 44a-44c show section views of an alternative embodiment of devices and methods of use according to the invention;

FIGS. 45a-45c show section and isometric views of an alternative embodiment of devices and methods of use according to the invention;

FIGS. 46a-46d show section views of an alternative embodiment of devices and methods of use according to the invention;

FIGS. 47a-47e show section views of an alternative embodiment of devices and methods of use according to the invention;

FIGS. 48a-48c show section views of an alternative embodiment of devices and methods of use according to the invention;

FIGS. 49a-49c show section views of an alternative embodiment of devices and methods of use according to the invention;

FIGS. 50a-50 show section views of an alternative embodiment of devices and methods of use according to the invention;

FIGS. 51a-51d show section views of an alternative embodiment of devices and methods of use according to the invention; and

FIGS. 52a-52c show section views of an alternative embodiment of devices and methods of use according to the invention.

### DETAILED DESCRIPTION OF THE INVENTION

The present invention provides a number of devices and methods for the efficient, effective, rapid and economic storage, shipment, moving and/or transferring, mixing, reacting, isolation, conditioning, lyophilization, stabilization, metering, dispensing, detecting, manipulation and management of materials, being particularly useful with small quantities of solids, powders and liquids.

The invention may be implemented in a variety of designs, as are represented in preferred embodiments by the following illustrative examples. The following examples are presented to illustrate the advantages of the present invention and to assist one of ordinary skill in making and using the same. These examples are not intended in any way otherwise to limit the scope of the disclosure.

In the following section, the initial examples describe a number of somewhat complex storage cell types and designs with techniques for fabricating and manipulating such cells. Later examples describe a number of detection techniques generally referenced in terms of a simple nominal cell type. However, per the invention, any cell type may potentially be used for any storage or reaction purpose with any detection technique. All such combinations of these discrete elements are included herein.

Devices with Integral Channels

### EXAMPLE 1

### Storage and Assay Sheet

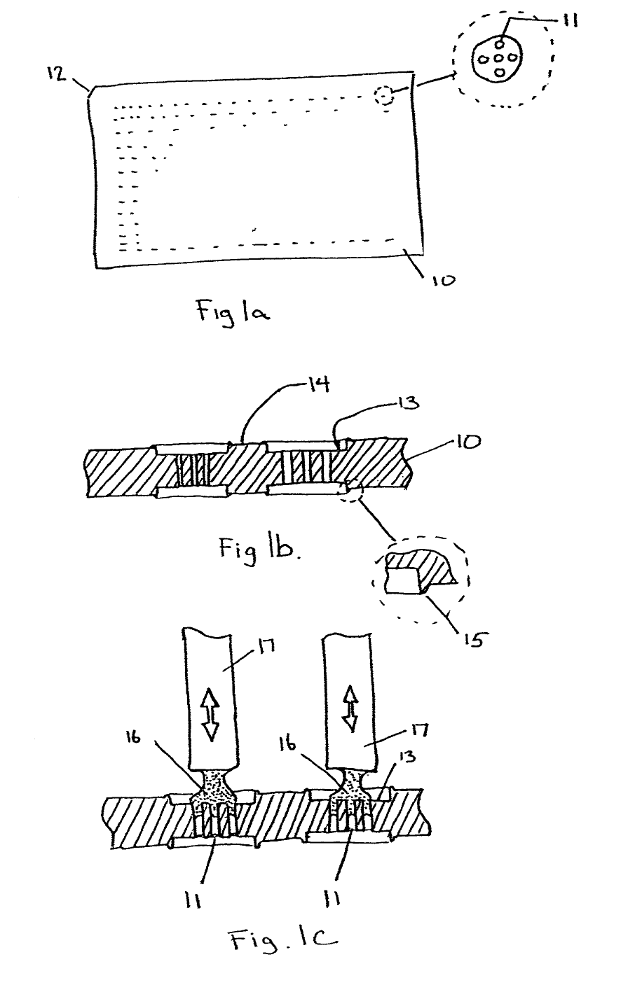

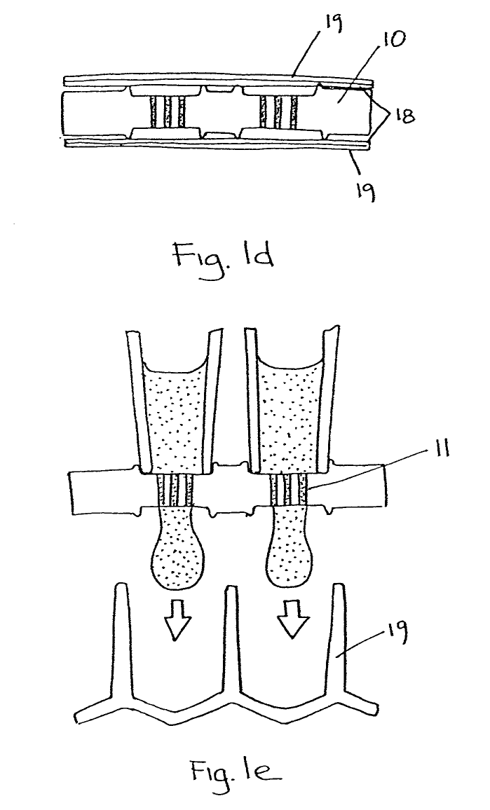

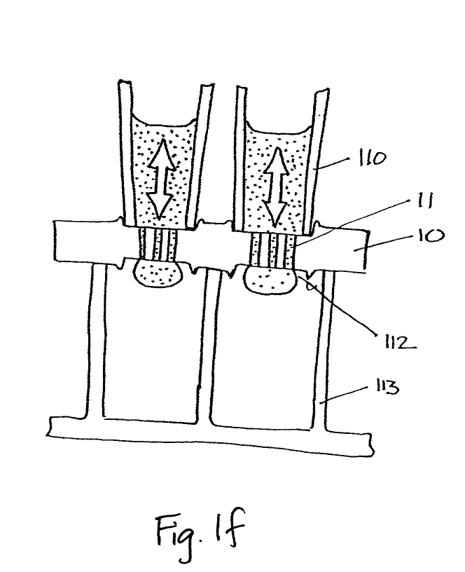

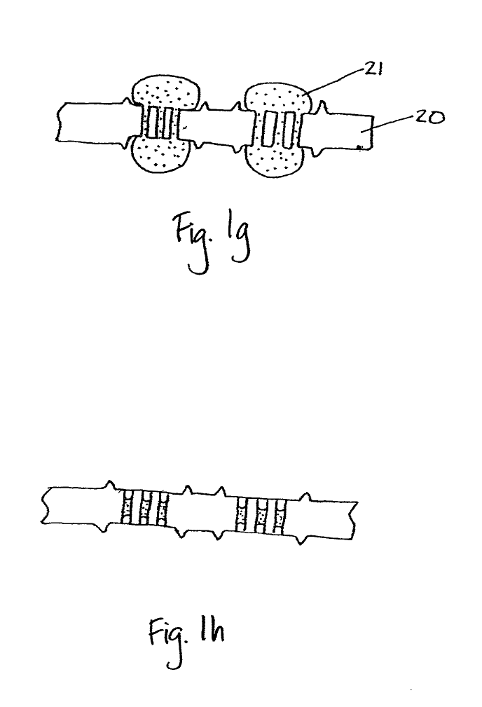

In one preferred embodiment as shown in FIG. 1a, a sheet 10 of solid plastic such as polypropylene is formed to contain 384 cells, each cell comprising a set of one or more holes in a closely spaced cluster, the cells being arrayed in a 16×24 grid to align with a standard 384 well microplate. The total available storage volume for each hole cluster cell is defined by the number of holes and their dimensions, which can be modified by changing the sheet thickness, number of holes per cluster, hole diameter or draft angle, plus the extent to which the holes can be filled.

Sheets may optionally contain locating features (physical and optical) and identification labeling such as sequential numbering or bar codes. In this example, one corner 12 of the sheet is chamfered to indicate proper location or orientation during use. Furthermore, a bar code may be printed on the sheet for identification, to designate a top surface and/or to encourage proper orientation.

Referring to FIG. 1b, two hole cluster cells, each comprising one or more holes, are shown in detailed section view for clarity. Each side of the sheet 10 may be optionally debossed around each hole cluster so that the sheet surface 13 immediately surrounding each hole is lower than the nominal surface level 14. This prevents sealing films or adjacent layers from contacting the stored sample, thus minimizing the likelihood of wicking cross contamination. Alternately, holes may be bidirectionally tapered, causing a sample to locate preferentially in the region of smallest diameter at the center of the sheet thickness (not shown).

Alternately, a small lip 15 may be formed around each hole cluster during the creation of debossed details. This lip may be used to concentrate ultrasonic or heat energy during sealing of the hole cluster with a seal, enhancing the quality of the seal in the local region. The lip 15 can also help to reduce the force required for seal removal by reducing the contact area of the seal, thereby potentially enhancing usability of this embodiment. Alternately, with adhesive sealing, the lip could optionally have a flat surface to facilitate sealing and also seal removal.

In FIG. 1c, liquid samples 16 are transferred to the hole clusters 11 with a simple or multi-channel pipette or pin transfer device 17. Liquid 16 is drawn into each hole cluster 11 by a combination of capillary force, gravity, liquid momentum or liquid pressure, with capillary forces predominating in a preferred embodiment. Hole geometry (diameter, height, draft angle, aspect ratio) and the extent of hole under/over filling are major factors for determining metered volume. Certain enhancements can be made to the sheet 10 to optimize fluid metering and positioning: these include, but are not limited to, surface modification of the inside of the holes 11 and/or the adjacent surface to increase hydrophilicity, or localized coating or modification of the surface 13 surrounding the holes to increase hydrophobicity. Such enhancements are to encourage the fluid samples to locate inside the holes rather than on the adjacent surface. Advantageously, the holes may be dimensioned such that capillary tension alone keeps the fluid inside, irrespective of gravitational force and the position of the sheet. Optionally, the transferred fluid samples 16 may be evaporated, dried or lyophilized before optionally being sealed as described below.

Referring to FIG. 1d, it can be seen that each side of sheet 10 may be sealed such as with a multi-layer adhesive or heat welded film for long-term storage of the samples. Advantageously, film layer 18 contacting sheet 10 may be polypropylene for its chemical resistant and sealing properties. Similarly, outside film layer 19 may be aluminum foil to minimize the transmission of oxygen, moisture and ultraviolet light. Alternatively, a single film may provide both functions. Multiple sheets 10 may be stored together in a cassette designed to interface with standard robotic retrieval equipment (not shown). Optionally, such cassettes may be placed in a freezer or refrigerator for storage at low temperatures to preserve sample integrity for extended periods.

Optionally, in preparation for an assay as shown in FIG. 1e, the seals are removed and the sheet is positioned above a microplate 19 or second sheet with embossed wells. Reagent and/or buffer, may be pipetted through the small hole clusters 11 and into corresponding wells in a standard microplate 19 or other assay platform. The dissolving stored solid material, or stored fluid, or combination thereof is thereby rinsed into the wells, for subsequent reactions and/or detection.

As shown in FIG. 1f, in preparation for an assay, the seals are removed and sheet 10 is positioned so that both sides of each hole cluster 11 are open and not near another surface. Stored fluid may then be transferred out of sheet 10. A pipette tip or similar device 110 containing reagent and/or buffer or other fluid is placed against one side of the sheet in alignment with a hole cluster 11. The fluid is applied and directed through each small hole cluster 11, until a quantity 112 may appear on the opposite side. A gasket detail or sheet material may be placed between the pipette tip and the surface to eliminate or reduce leakage. Pressure on the pipette tip may provide a seal that causes the fluid to go through the hole cluster 11. Alternately, the tip may seal directly against the sheet or to a feature molded on the sheet as may be designed to assist with such sealing.

If the drop extrudes, the drop size may advantageously be small enough to remain attached to the opposite side of the sheet 10 such as by surface tension. The maximum drop volume will depend on a combination of surface energy characteristics, geometry and orientation with respect to gravity. The pipette may then optionally cycle the fluid volume back through hole cluster 11, one or more times to improve mixing. This process may be repeated as necessary to ensure adequate dissolution and mixing. The stored solid material, or stored fluid, or combination thereof is thereby mixed, before transfer to a separate plate, well or surface for chemical reaction and/or detection, or for reaction and/or detection on or within sheet 10 itself. Alternately, additional fluid may be added to each hole cluster 11 such that the overall fluid volume within the hole cluster cell in sheet 10 may be detached from the surface into a receptacle, to fall, e.g., by gravitational, pneumatic, inertial or hydraulic force or impulse. The support means 113 is optional, and if present may optionally also serve to collect and/or hold detached liquid. Alternately, sheet 10 containing cells 11 with fluid may be otherwise supported such as by the edges and positioned in any orientation. Alternately, fluid at the pipette tip may be placed in contact with the stored fluid or other material within the cell, and may be cycled in and out of the tip but not through the cell. Mixing and diffusion will occur during cycling and a seal around the pipette tip may not be required.

As shown in FIG. 1g, mixed fluid may be left in the area of each hole cluster by removing the pipette from contact with the surface. The volume left may be smaller or the same as the original stored volume, but may also be advantageously larger as illustrated, in which case it may remain attached to both surfaces of the sheet around the hole clusters 11, e.g., by surface tension. Furthermore, a chemical reaction may be caused to take place, and the detection of reaction products or unused reagents may be effected as is described in later examples. Per FIG. 1h, the cells comprised of hole clusters alternately may be significantly underfilled, or alternately the holes may be approximately exactly filled such that the fluid surface is approximately coplanar with the surfaces of sheet 10 (not shown).

After the complete experimental process as outlined above, the sheet 10 and pipette tips 110 may be disposed of such that all potential contamination and/or biohazard may be removed without contamination of the apparatus, instrumentation or robotic equipment.

Although this example uses a 384 well plate, a 96 or 1536 well plate may be used equivalently, as could other microtiterplate templates or other ensembles of any number of storage cells of any dimensional layouts or patterns, all of which are included in the present invention.

### EXAMPLE 2

### Extruded Nozzle Tips

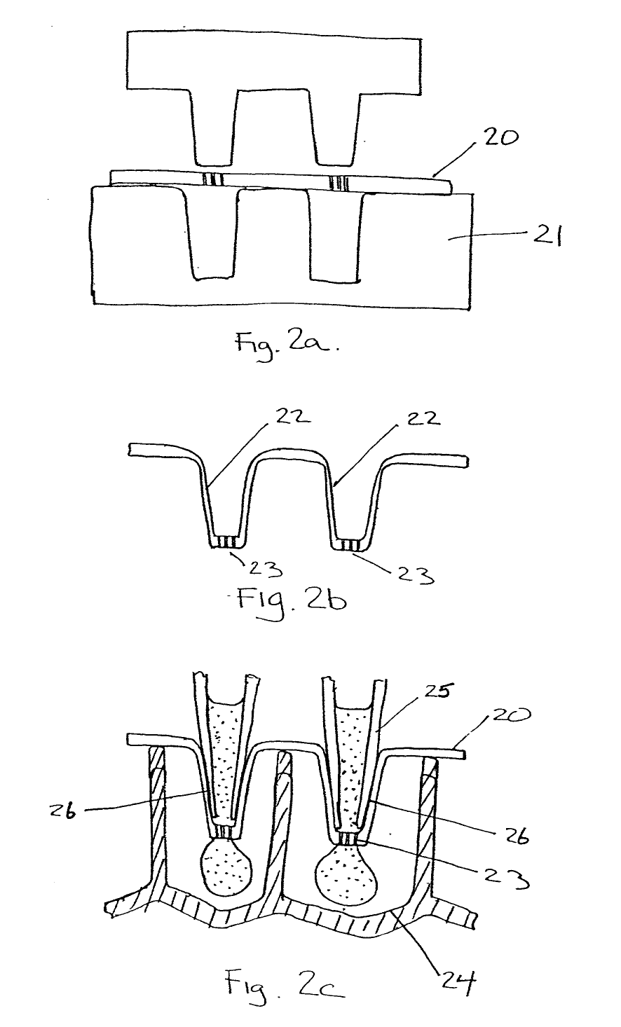

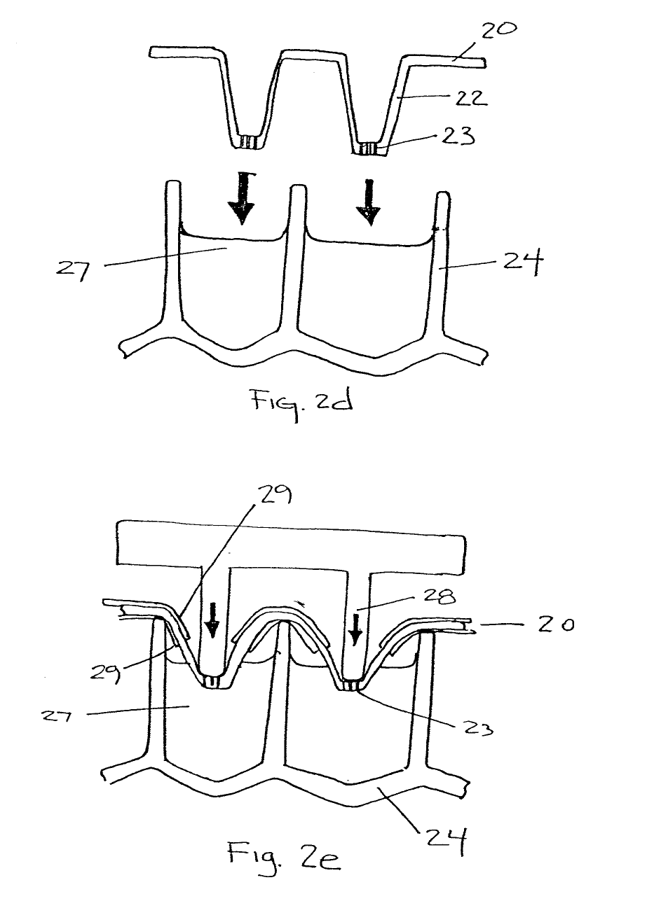

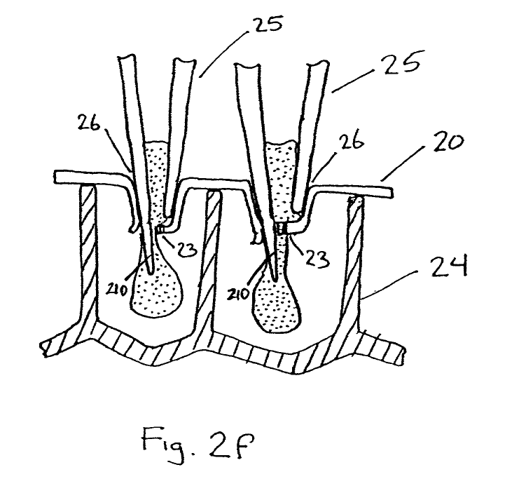

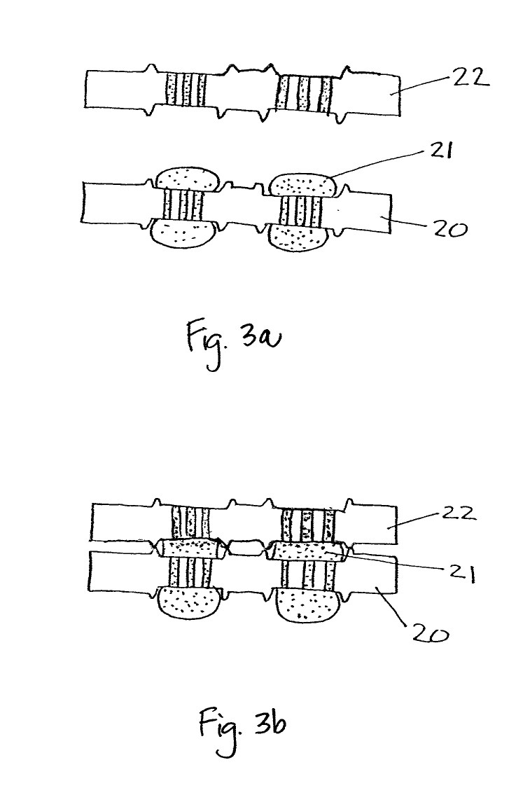

A sheet similar to that described above is loaded and sealed in the same manner. In preparation for an assay, the seals are removed and the pre-loaded sheet 20 is formed in a die 21, as shown in FIG. 2a. Per FIG. 2b, the die is used to extrude the area around each hole cluster 23 into a nozzle shape 22, to have the hole cluster cell areas of the sheet project away from the other portions of the sheet. This nozzle shape is designed to interface with pipette tips on the top and to direct fluid flow on the bottom. This will improve dispensing accuracy and minimize cross-well contamination.

This embodiment can be used in at least two ways. First, after extrusion of the nozzles 22 around each hole cluster 23 containing fluid, sheet 20 is positioned over a corresponding assay plate 24 as shown in FIG. 2c. A multi-channel pipette device 25 is lowered into the extruded nozzles, forming a wedge-seal 26 around each pipette tip. Reagent and/or buffer is dispensed through the hole clusters 23, taking the dissolving solid and/or stored fluid with it into each corresponding assay plate well 24. The sheet 20 is then removed from the pipette tips and disposed of, or ejected with the pipette tips.

The second method adds the dissolving solid and/or stored fluid to wells pre-filled with reagent and/or buffer. After extrusion of the nozzles 22 around each hole cluster 23 containing material, the sheet 20 is positioned over a corresponding assay plate 24, as shown in FIG. 2d. The sheet 20 is then lowered until it rests on the assay plate 24. The extruded nozzles 22 contact the surface of the reagent and/or buffer, transferring the stored material from the hole clusters 23 to the corresponding assay plate wells 24. The sheet may be disposed of after use, or optionally may be thoroughly cleaned for reuse.

Alternatively, the nozzles may be extruded with the sealing film or foil still in place. This technique depends on differing ultimate strengths of the sealing film or foil and the sheet. The sealing film material is selected and design is configured for this to tear well before the sheet during nozzle extrusion, thereby leaving an open path for fluid transfer. Per FIG. 2e, a sheet 20 is placed on an assay plate 24 that has been prefilled with reagent or buffer 27, and on which the corresponding wells align. An array of probes 28 extrudes the regions of the sheet 20 surrounding each hole cluster 23. The sealing film 29 tears first while the sheet 20 stretches until contacting the fluid in the prefilled assay plate 24, transferring stored fluid and/or dissolving material from the hole clusters 23. Alternatively, two different types of sealing film 29 maybe advantageous used whereby the lower film layer ruptures while the upper film layer remains intact (not shown) such that the hole clusters contact the reagent or buffer 27 while probes 28 remain uncontaminated by either the hole cluster contents or fluid 27.

Another embodiment, shown in FIG. 2f, is similar to that of FIG. 2c, with the addition of needle-like tips 210 at the end of the pipette tips. The areas of the sheet 20 around each hole cluster 23 containing fluid are dimpled or extruded to form short nozzles. Sheet 20 is positioned over the corresponding assay plate 24. A multi-channel pipette device 25 is lowered into the extruded nozzles, forming a wedge-seal 26 around each pipette tip. Reagent and/or buffer are dispensed through the hole clusters 23, taking the dissolving solid and/or stored fluid with it into each corresponding assay plate well 24. The needle-like tips on the end of the pipette tips 25 guide the drops into the wells, facilitating accurate dispensing. The sheet 20 may then be removed from the pipette tips and disposed of, or ejected with the pipette tips.

### EXAMPLE 3

### Sample Transfer and Assay by Sheet Contact or Displacement

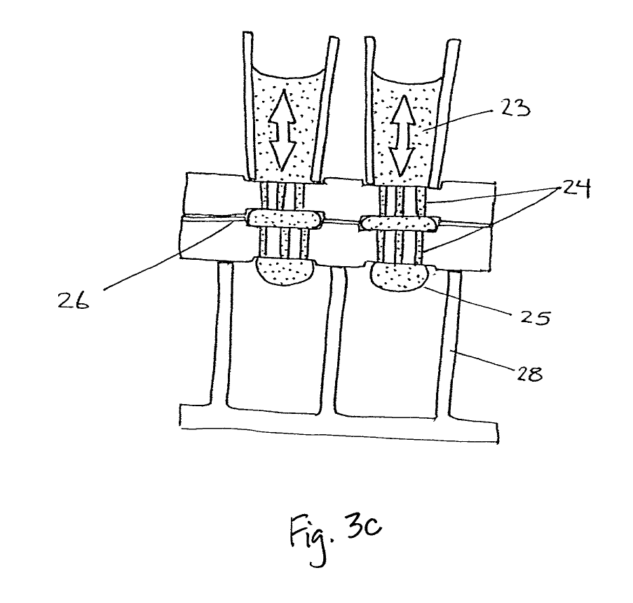

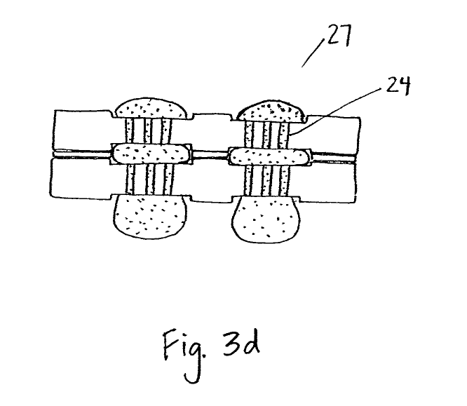

In FIG. 3a reagent and/or buffer or other fluid or gel or other material 21 is placed in wells on a custom assay sheet, in small drops on a flat sheet or surface, or in a storage and transfer sheet 20 similar to those described in Example 1.

In FIG. 3b, a second storage and transfer sheet 22 pre-loaded with fluid or solid samples is prepared by removing the bottom seal or both seals, if used, and is placed over the bottom sheet 20, aligning each sample storage location with a well or drop 21 on the first sheet. Either sheet 20 and/or sheet 22 may have holes overfilled, underfilled or approximately exactly filled with fluid, or at least one may contain material in dried, lyophilized or gel form. The second sheet 22 is brought into contact with the reagent and/or buffer 21, transferring the dissolving solid material if present and/or stored fluid resulting in optional dissolution of solids and diffusional mixing, and optionally commencing subsequent chemical reactions. This technique enables many assays to be performed in parallel without the use of pipetting equipment.

Alternately, per FIG. 3c, seals on both sides of each sheet may be removed, so that when additional fluid 23, such as a reagent and/or buffer solution, is pipetted to each hole cluster, excess fluid passes through both sheets until a drop 25 may appear on the opposite side. Optionally, providing means to prevent wicking and/or cross contamination between the sheets and/or cells may enhance performance. Such means could, by examples, comprise seal or gasket 26, or hydrophobic surface treatment causing the fluid to stay within the hole cluster, preventing cross-contamination of other cells.

The pipette may then optionally be used to cycle fluid back through each hole cluster 24 of juxtaposed cells, optionally repetitively, to encourage mixing, which may be repeated as necessary. Stored solid material, or stored fluid, or combination thereof is thereby mixed, ready for optional transfer to a separate plate or for surface for reaction and detection, or for subsequent reactions and/or detection on or within the sheet itself. The support 28 may optionally be used to support the sheets during one or more of the steps above and optionally to collect detached fluid, or, alternatively, the sheets may be otherwise supported such as at the edges and optionally positioned in any orientation.

Per FIG. 3d, mixed fluid 27 may be left in the area of each hole cluster 24 after removing the pipette from contact with the surface. The volume left may be smaller or the same as the original stored volume, but may also be advantageously larger. In this case, the mixed fluid may remain attached to both surfaces of the sheet around the hole clusters such as by surface tension. A chemical reaction may now be effected within the fluid volume, and detection of reagents or products thereof may be effected as described in a later example.

This example may be extended by stacking more than two sheets together, such that more than two storage hole cluster cells may be juxtaposed and their contents mixed and optional solid material dissolved in the liquid from other juxtaposed cells, or additional fluid may be added by a pipette or similar means.

### EXAMPLE 4

### Assay within Storage Cell

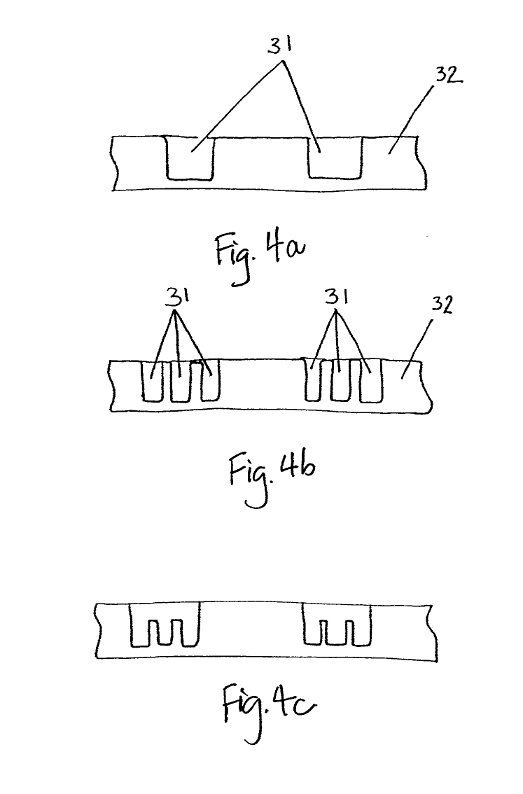

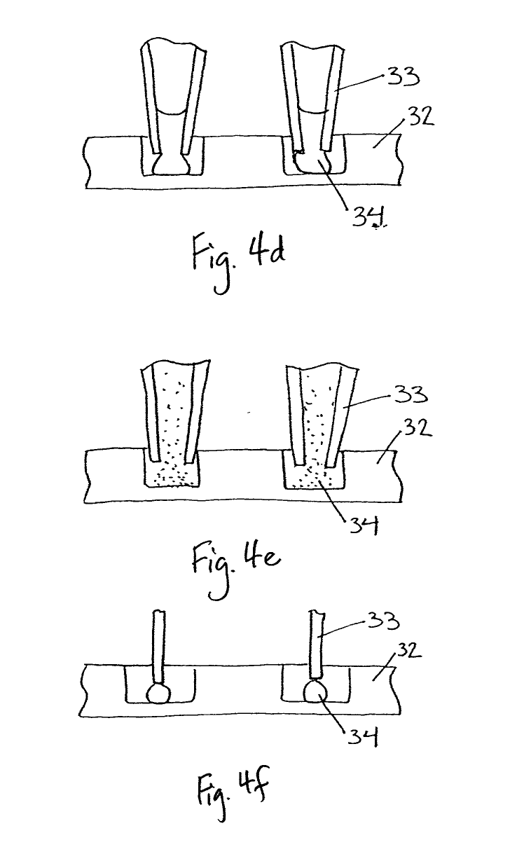

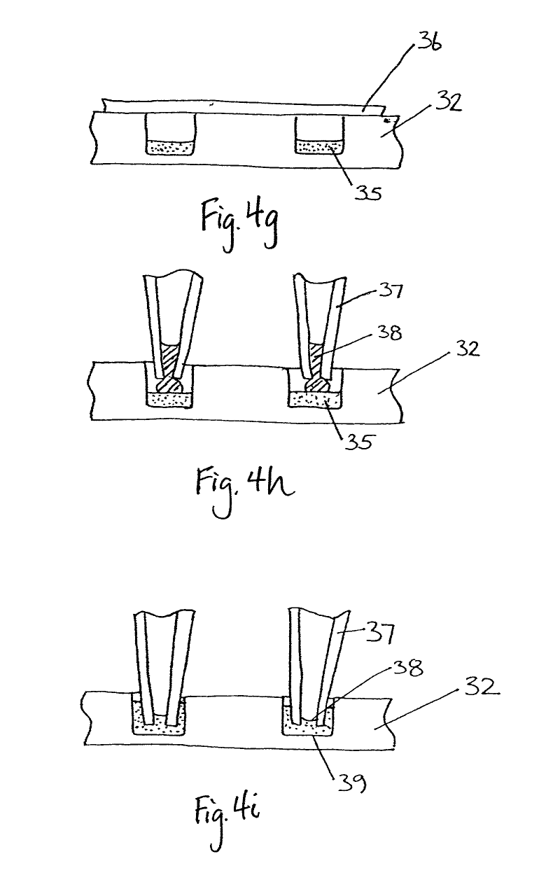

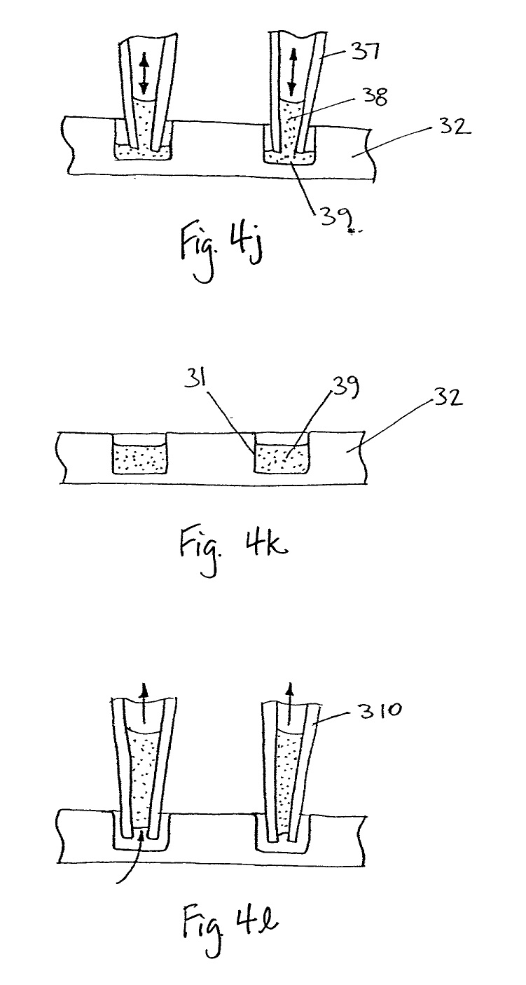

FIGS. 4a-4c depict similar storage cells 31 formed in sheet 32 similar to those described above, except that such cells are formed in the format of a well. Various formats of such cells may be formed, such in FIG. 3a where a simple hollow cavity is formed, or such as in FIG. 3b representing a number of cavities such as may optionally be interconnected, or such as in FIG. 3c where two or more cavities (which may have walls between them of height less than the sheet thickness), or other arrangements as may be configured to hold material therein. Per FIGS. 4d -4f, material such as a fluid, solid, powder gel or slurry 34 may be deposited within the cell such as by a pipette or pin transfer device 33, and then optionally evaporated, dried or lyophilized, and then optionally sealed by means including those outlined above 36 to form a sealed cell such as 35 per FIG. 4g. After one or more such cells in the sheet have been so prepared, one or more sheets may be positioned in a storage cassette and optional stored under cooled or cryogenic conditions to assist with material stability.

After removal of sheet 32 from storage, cell 35 may be unsealed as above. Then, per FIG. 4h, fluid 38 such as chemical reagents or buffer may be added such as by pipette 37 or by other means. Per FIG. 4i, fluid 38 dispensed from pipette tip or similar 37 mixes with and dissolves the material 35 which had been stored in the cell 31. Advantageously, the fluid addition may cause mixing and optional solution of the stored material, such as by causing turbulent flow. Optionally, per FIG. 4j, the combined fluid and stored matter 39 may be retracted into the pipette tip 37 and expelled again one or more times to assist with mixing and solution. Thus, per FIG. 4k, after addition of fluid 38, and optional retraction/expulsion cycles as above, mixed and dissolved fluid 39 containing the stored material 35 is contained within cell 31 within sheet 32. Optionally, the mixed fluid 39 may then undertake a chemical reaction, optionally assisted by heat, light or other known means, whose occurrence, products or unreacted reagents may then be detected and/or measured within the cell or elsewhere.

Per FIG. 4l, the mixed fluid 39 may be removed from cell 31 within sheet 32 by pipette 310 or other means, such as for chemical reaction and/or detection or other purposes elsewhere. Alternately, the mixed fluid 39 may remain in cell 31 for optional in-situ reaction and/or detection and/or other analytical purposes or for one or subsequent chemical reactions and/or processes.

As above, the arrangement of storage cells 31 and sheet 32 may be in standard 96, 384 or 1536 well formats to be compatible with existing instrumentation and/or robotic and/or detection equipment, or any other dimensional arrangement or pattern may be employed.

Following completion of this process, sheet 32 and pipette tips 33, 37 and 310 may be disposed of and all potential contamination/biohazards removed without contamination of the apparatus, instrumentation or robotic equipment.

### EXAMPLE 5

### Assay Utilizing Stacked Storage Cells

This method is a combination of examples previously described.

FIG. 5a depicts storage sheet 42 containing one or more bottomless cells 43 optionally containing material deposited therein 45 that may optionally have been previously sealed, stored and unsealed. Positioned against sheet 42 is sheet 41 containing one or more storage cells 46 optionally containing liquid material 47 optionally stored beforehand per Example 1 above, such that one or more cells 43 are juxtaposed against matching cells 46 with optional sealing means between as outlined above. FIG. 5b indicates an alternate configuration whereby stored material 47 is solid or dried material such as adhered to the interior walls of storage cell 46. Pipette or other fluid dispensing device 48 is positioned to dispense fluid 49 into the stacked cells 43 and 46.

Per FIG. 5c, pipette 48 or other means is used to add fluid 49 optionally containing chemical reagents or buffer to stacked cells 43 and 46 causing the fluid and stored materials to be mixed within the cell . As above, this fluid may be added by turbulent flow to assist with mixing and/or solution, and per FIG. 5d, the mixed fluid 410 may be cycled into and out of the pipette 48 one or more times to assist with mixing and solution. Per FIG. 5e, the mixed fluid 410 may be deposited within stacked cells 43 and 46 such as for a chemical reaction to take place, optionally assisted by heat, light or other known means, and/or for detection of the occurrence of one or more chemical reactions, or for the detection and optional measurement of one or more products of chemical reactions. Optionally, a chemical reaction may occur and the occurrence of this reaction may be detected, and one or more reagents or reaction products may be detected and/or measured, or one or more additional substances may be added for other purposes. Alternately, the mixed fluid may be transferred out of the stacked storage cells to another location by pipette or other means, for optional reaction and/or detection and/or measurement or other purpose or purposes elsewhere.

This example may be extended by the use of two or more storage cells per Example 1 above stacked against a closed bottom cell per Example 4 above, to provide for the mixing and/or solution and/or chemical reaction of one or more materials optionally supplemented by added reagents and/or buffer solution.

As above, the arrangement of storage cells and sheets may be in standard 96, 384 or 1536 well format to be compatible with existing instrumentation and/or robotic and/or detection equipment, or any other dimensional arrangement or pattern may be employed.

After the complete experimental process as outlined above, the sheets 41 and 42 and pipette tips 48 may be disposed of such that all potential contamination and/or biohazard may be removed without contamination of the apparatus, instrumentation or robotic equipment.

### EXAMPLE 6

### Integrated Assay Device

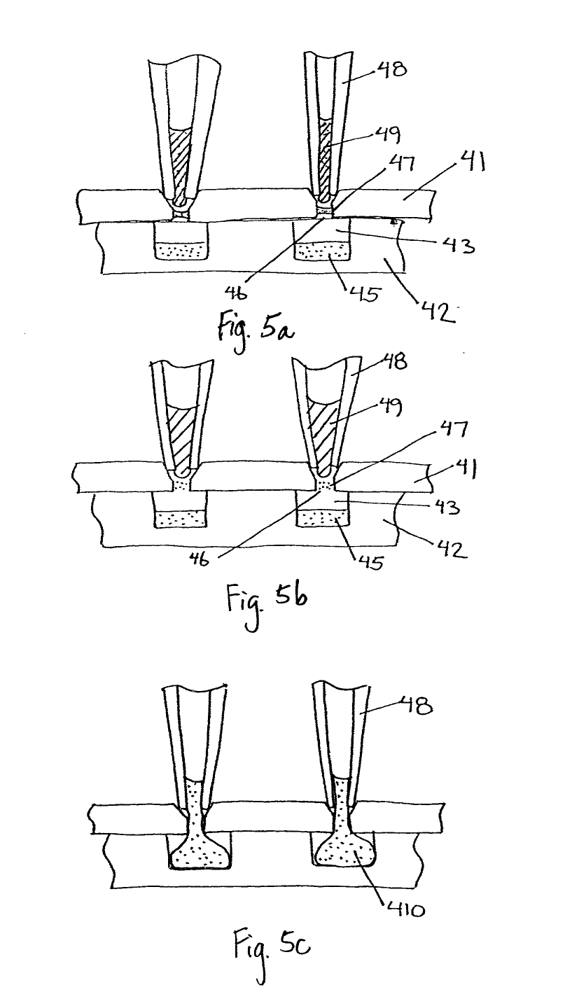

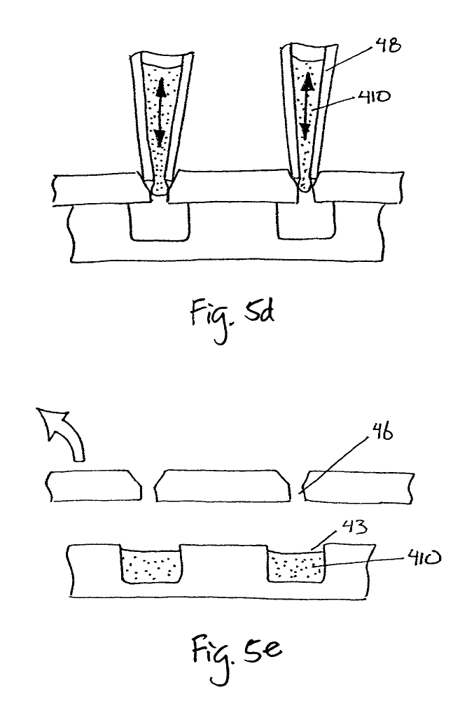

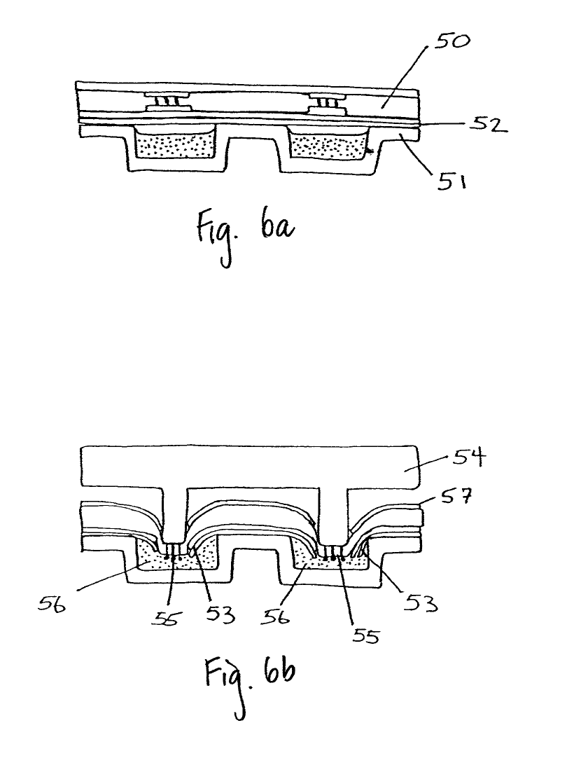

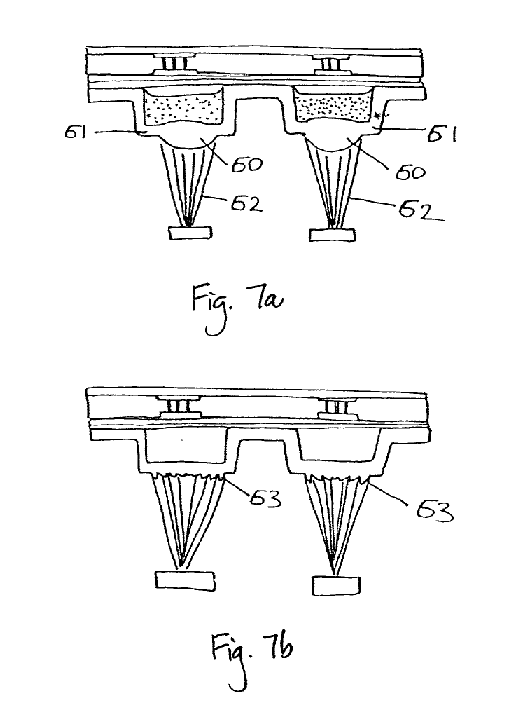

An integrated assay device, shown in detailed section view in FIG. 6a, combines a storage and transfer sheet such as described above, juxtaposed with a corresponding sheet containing reagent and/or buffer. For many assays, the stored volume of reagent and/or buffer may be several to 100 times the volume of each cell in a storage and transfer sheet.

A storage and transfer sheet 50 is loaded and sealed as described above. A reagent sheet 51 is loaded and sealed, then placed together with the storage and transfer sheet 50 so that the corresponding cells align. Next, the two halves may be fastened together by mechanical, adhesive or other means 52.

To perform the assay, the samples stored in the top half of the device are forced to mix with the reagent and/or buffer in the lower half. In a preferred embodiment, as shown in FIG. 6b, the seals are ruptured as a die 54 extrudes each hole cluster 55 into a corresponding cell 56 containing reagent and/or buffer. This brings the fluids into contact, optionally starting the subsequent chemical reaction. Advantageously, the upper sealing layer 57 may remain intact such that the die tips are not contaminated with hole cluster contents or with reagent or buffer.

### EXAMPLE 7

### Integrated Assay Device with Enhanced Detection Features

Optical detection is commonly used for a large number of chemical assays. The integrated assay device described in Example 6 can include features to enhance detection sensitivity and discrimination between cells.

As shown in FIG. 7a, a molded-in lens 60 in each cell wall 61 focuses or directs light emissions 62, increasing detection sensitivity while reducing stray light and possible cross-contamination of results. The cell wall and lens material must transmit the optical wavelengths of interest at acceptable efficiency levels. This lens may take the form of a convex lens, or Fresnel type elements 63 could be used in a flatter package as shown in FIG. 7b. Other optical elements such as diffraction gratings, filters, half-silvered surfaces, mirrors, etc. (not shown) could be incorporated in the device to focus, filter or otherwise modify light emissions, as will be apparent to someone skilled in the art.

### EXAMPLE 8

### Multi-Sample Cells

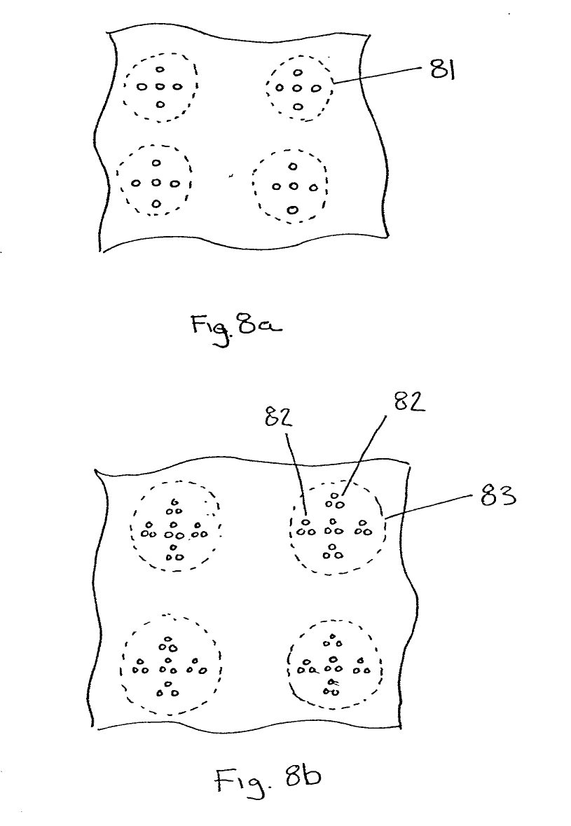

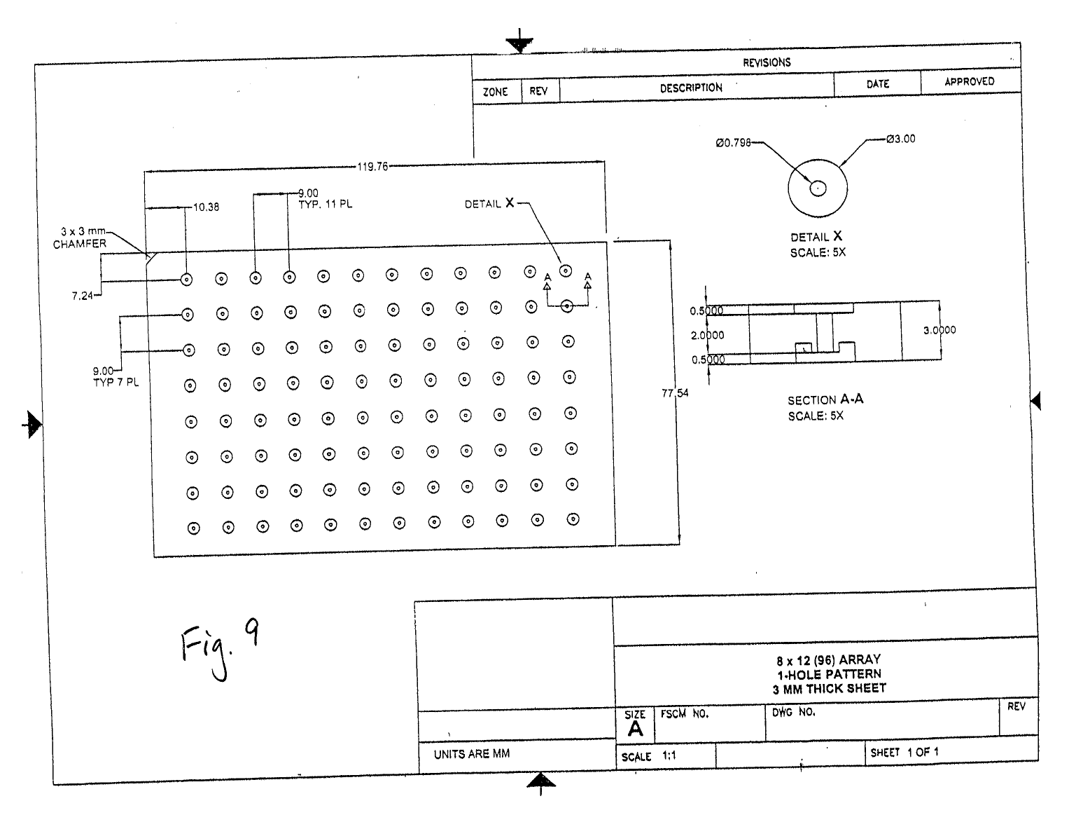

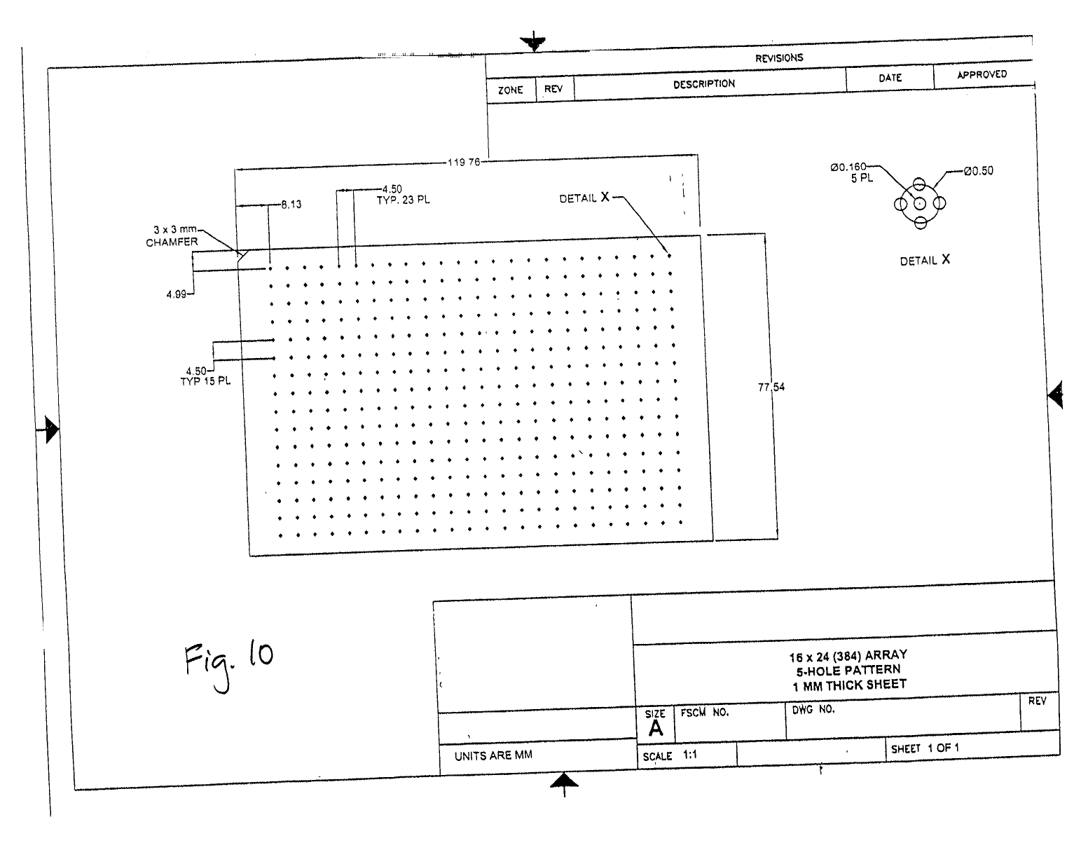

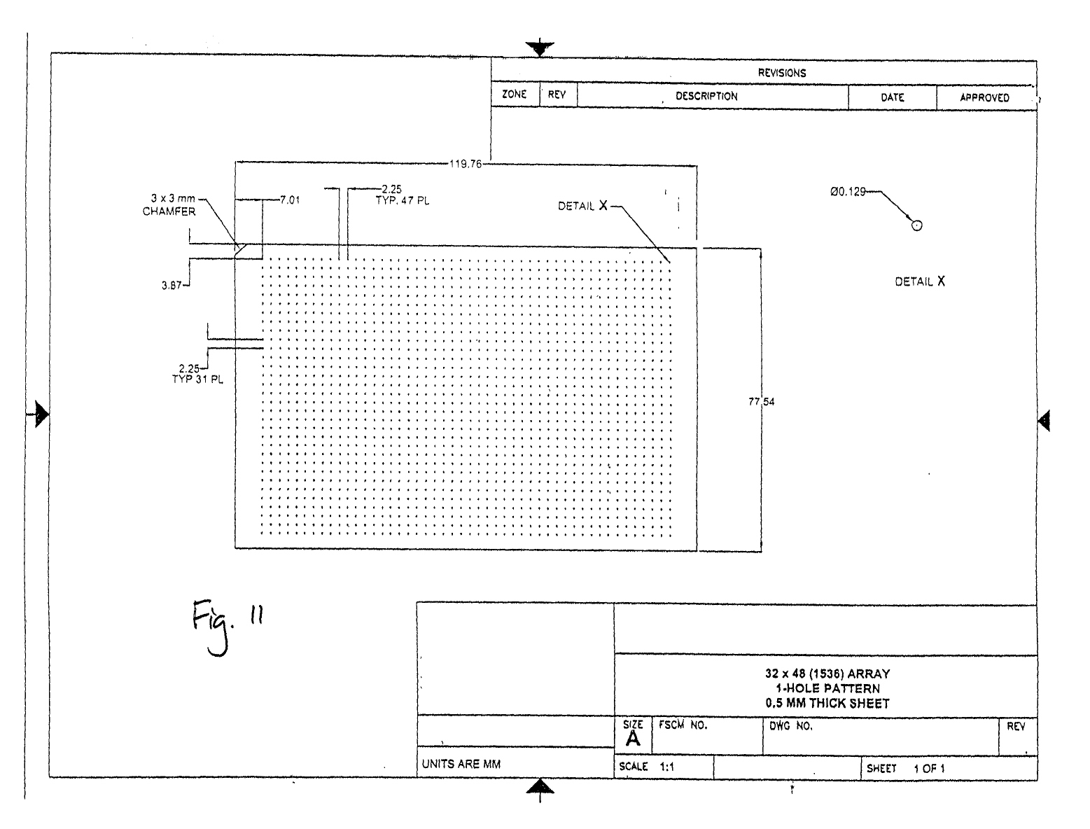

It may be desirable in some cases to test multiple samples per cell. In this way, the number of initial tests required to identify e.g. a drug candidate may be reduced. FIG. 8a shows a detail of a sample storage and transfer sheet similar to the one described in Example 1. Each hole cluster 81 can contain a number of holes to define a storage cell. Typically, all holes within a cluster 81 would be filled at the same time with one sample. However, in this alternative example, different holes within each cluster may be loaded with different samples.

FIG. 8b shows a detail of a different configuration. Here, mini-clusters of holes 82 are arrayed in full clusters 83, which are part of a larger array. Each mini-cluster hole 82 can be loaded with a different sample. Alternately, some mini-cluster holes may contain the same sample, to provide storage of a larger quantity of this sample within the storage capacity of the entire full cluster. Thus, assays of differing intrinsic sensitivities may advantageously have their effective sensitivities normalized, compensated or made more similar by providing different quantities of the appropriate samples within a full cluster.

During sample transfer as is described in previous examples, each full cluster 83 is transferred as a cell or group, for example to an assay plate. The different samples contained in each mini-cluster hole 82 are tested simultaneously in a parallel or multiplexed manner. If a positive result occurs, the samples may then be tested separately and individually.

### EXAMPLE 9

### Design for 96 Cell Embodiment

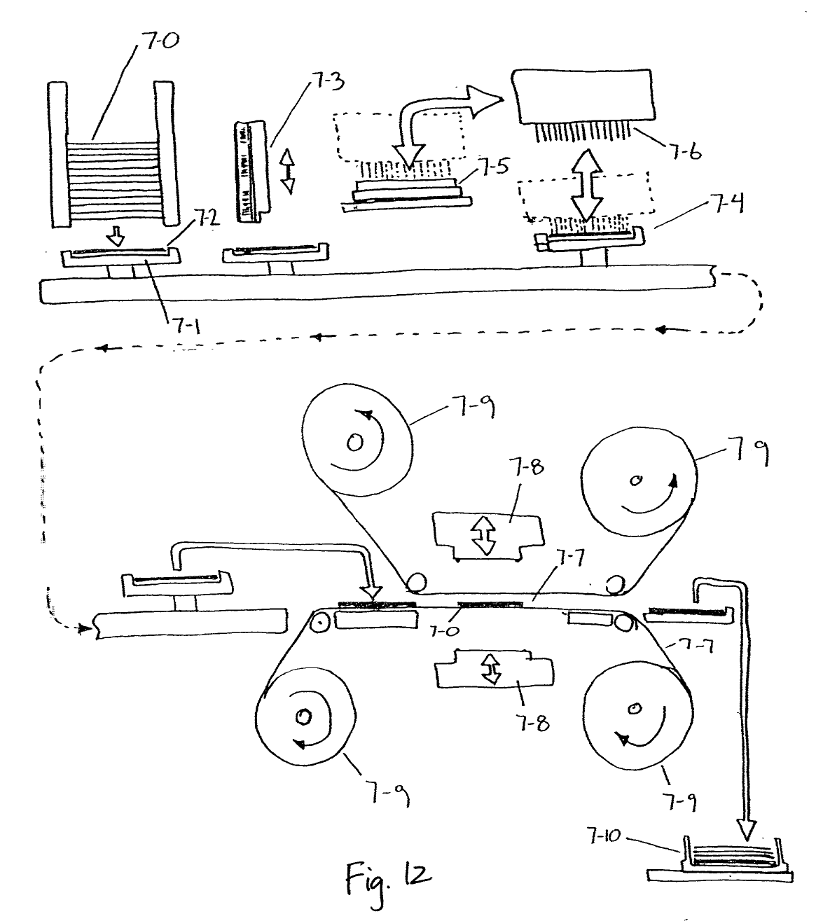

FIG. 9 depicts a CAD drawing for an actual embodiment and design for an array of storage cells formed in a sheet as described above. In this design, the storage cells are laid out in an 8×12 array providing 96 such storage cells in a format optimized for use with existing 96 well microtiter plates, etc.

### EXAMPLE 10

### Design for 384 Cell Embodiment

FIG. 10 depicts a CAD drawing for an actual embodiment and design for an array of storage cells formed in a sheet as described above. In this design, the storage cells are laid out in a 16×24 array providing 384 such storage cells in a format optimized for use with existing 384 well microtiter plates, etc.

### EXAMPLE 11

### Design for 1536 Cell Embodiment

FIG. 11 depicts a CAD drawing for an actual embodiment and design for an array of storage cells formed in a sheet as described above. In this design, the storage cells are laid out in a 32×48 array providing 1536 such storage cells in a format optimized for use with 1536 well microtiter plates, etc.

### EXAMPLE 12

### Apparatus for Loading and Sealing Devices

When multiple sheets are loaded, major efficiencies may be realized by automating the loading and sealing process. FIG. 12a illustrates one embodiment of such an automation apparatus. Fabricated empty sheets 70 are stacked in a cassette to enable continuous feeding of sheets. For each cycle of the apparatus, a sheet is transferred to a tray 71 or conveyor by direct transfer, robotic arm or other means. The tray contains locating features such as pins, walls or a mating receptacle 72 to ensure precision placement of the sheet. Since the sheets are typically fabricated from thin material, a clamping mechanism such as a frame or vacuum could be employed to maintain precision placement (not shown).

A sequential numbered label or bar code 73 can be applied at this time to enable automated subsequent identification of the sheet and contained samples. Numerous labeling techniques and codes are known to those skilled in the art.

The tray or conveyor moves sheet 70 to transfer station 74, where fluid is transferred from a multi-well storage plate 75 or other container to the sheet. Typically, a multi-tip pipette or pin transfer device 76 aspirates or picks up a quantity of each sample from a multi-well storage plate 75 or other container, then moves over the sheet 70, and transfers some or all the sample from each pipette or pin to each cell.

The transfer device 76 may advantageously use the same array and number of tips or pins as the arrangement of cells on the sheet 70. Alternately, it may contain an even fraction of the number of cells, so that several transfers from different storage plates are required to load one sheet. Depending on the amount transferred, the multi-tip pipette or pin transfer device may need to return to the storage plate or container for each sheet, or may load several sheets in sequence. To minimize cross-contamination, pipette tips or pins are discarded or thoroughly rinsed between storage plate changes. Other sample transfer techniques may optionally be used, such as piezo-type dispensing or mechanical transfer of beads, as are known to those skilled in the art.

Next, a sheet is moved by conveyor, robotic arm or other means to a sealing station. Here the sheet is positioned between upper and lower sealing films 77. A die 78 brings the films into contact with the sheet 70, and applies heat, pressure or other means, sealing the top and bottom surfaces of the sheet 70. For some devices, only one seal will be required, and others may not require sealing. The sealing sheet or sheets can be trimmed or die cut at the same time as sealing occurs or separately. Sealing sheets may be precut, or fed from rolls 79 and cut at the time of sealing.

Once sealed, the sheet is moved by conveyor, robotic arm or other means to a storage cassette 710. Typically, several sheets of the same sample array will be made, and stored in one or more cassettes. The cassette may be advantageously designed to use the same footprint as existing assay plates, to enable existing automation equipment to be used. Once the cassette contains the desired number of sheets, a removable lid may be applied, if desired (not shown) optionally after filling with dry or inert gas. The full cassette is removed by conveyor, robotic arm or other means and may be replaced by another.

For many samples, exposure to moisture, light, temperature extremes or oxygen will cause degradation. The sheets and stored samples described above may advantageously be housed in a storage or transfer enclosure (not shown). The enclosure may be positively pressurized with a neutral gas such as nitrogen or argon, and the internal temperature, humidity and exposure to light can be controlled by means known to those skilled in the art.

Although the above example is expressed as a set of discrete components and actions, many alternative combinations of apparatus elements and discrete steps in the overall process are possible. All such alternate elements and combinations, as provide all or some of the above functionality or similar, will be apparent to those skilled in the art and are all covered by the present invention.

### EXAMPLE 13

### Performance Testing

Four devices as described in the above example were fabricated, and tested to demonstrate performance. Each device contains four cell clusters designed to store a maximum of 100 nL of fluid per cluster. Testing as described below was performed to measure the coefficient of variability.

A 1% solution of fluorescein dye in DMSO was prepared. A stainless steel pin tool was used to manually transfer approximately 50 nL of this solution to each cell cluster. A manual pipette tip containing 2 microliters of aqueous buffer solution was used to transfer the contents of each cell cluster to a designated microplate well as follows.

The pipette tip containing the buffer solution was aligned with the cell cluster and brought into contact with the top surface of the device. The buffer solution was dispensed and re-aspirated through the cell cluster three times to ensure mixing. The mixed contents were aspirated into the pipette tip and dispensed into an empty microplate well. This sample was diluted with 98 microliters of distilled water.

This process was repeated in triplicate for each of the four cell clusters on each of four devices. Once all samples were transferred to the microplate and diluted, the plate was read in a fluorescence microplate reader.

In addition to the samples, plain water was pipetted directly to 12 wells as a control. A measured dilution of the fluorescein dye was prepared and pipetted into microplate wells to simulate 100% transfer. Further, the 1% solution was transferred by stainless steel pin tool directly to 4 empty microplate wells, then diluted to determing the variability introduced by the loading method alone.

Qualitative results are favorable. It was shown that sample reliably loaded into and unloaded from each cell cluster. This was confirmed by looking for the presence of the colored solution. Quantitative results shown in TABLE I support this observation. In all cases, luminescence values are considerably higher than plain water, indicating that sample was transferred from each cell cluster to its corresponding microplate well. The coefficient of variability (%CV) in this test was approximately 39%.

TABLE I

| | Sheet 1 | | | Sheet 2 | | | Sheet 3 | | | Sheet 4 | | |
|---|---|---|---|---|---|---|---|---|---|---|---|---|
| Site A | 0.282 | 0.207 | 0.250 | 0.142 | 0.208 | 0.129 | 0.129 | 0.144 | 0.195 | 0.096 | 0.262 | 0.237 |
| Site B | 0.222 | 0.256 | 0.252 | 0.219 | 0.127 | 0.126 | 0.075 | 0.121 | 0.170 | 0.054 | 0.153 | 0.148 |
| Site C | 0.178 | 0.226 | 0.225 | 0.166 | 0.129 | 0.217 | 0.153 | 0.129 | 0.297 | 0.180 | 0.227 | 0.242 |
| Site D | 0.297 | 0.349 | 0.255 | 0.258 | 0.141 | 0.286 | 0.059 | 0.084 | 0.065 | 0.067 | 0.251 | 0.135 |

| | | | | | | | |
|---|---|---|---|---|---|---|---|
| Sheet 1 mean | 0.250 | Sheet 2 mean | 0.179 | Sheet 3 mean | 0.135 | Sheet 4 mean | 0.171 |
| SD | 0.043 | SD | 0.054 | SD | 0.064 | SD | 0.070 |
| % CV | 17.2 | % CV | 30.2 | % CV | 47.1 | % CV | 41.2 |

| Mean | 0.184 | | | | | | | | | | |
|---|---|---|---|---|---|---|---|---|---|---|---|
| SD | 0.072 | | | | | | | | | | |
| % CV | 39.2 | | | | | | | | | | |
| Controls | | | | | | | | | | | |
| Pin transfer 1% solution direct to dry well, then dilute, 4 wells | | | | | | | | | | | |
| 0.063 | 0.085 | 0.080 | 0.107 | | | | | | | | |
| Mean | 0.084 | | | | | | | | | | |
| SD | 0.016 | | | | | | | | | | |
| % CV | 18.8 | | | | | | | | | | |
| Measured dilution, pipette 100 microliters, 4 wells | | | | | | | | | | | |
| 0.306 | 0.320 | 0.296 | 0.297 | | | | | | | | |
| Mean | 0.305 | | | | | | | | | | |
| SD | 0.010 | | | | | | | | | | |
| % CV | 3.2 | | | | | | | | | | |
| Plain Water, pipette 100 microliters, 12 wells | | | | | | | | | | | |
| 0.024 | 0.025 | 0.024 | 0.027 | 0.024 | 0.024 | 0.025 | 0.024 | 0.025 | 0.025 | 0.026 | 0.028 |
| Mean | 0.025 | | | | | | | | | | |
| SD | 0.001 | | | | | | | | | | |
| % CV | 5.0 | | | | | | | | | | |

Temperature Control

### EXAMPLE 14

### Temperature Controlled Reactions

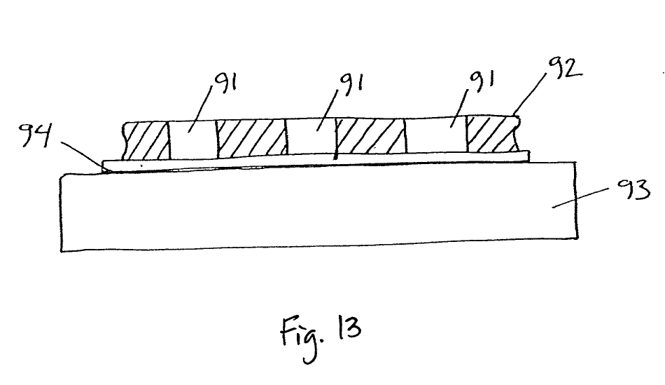

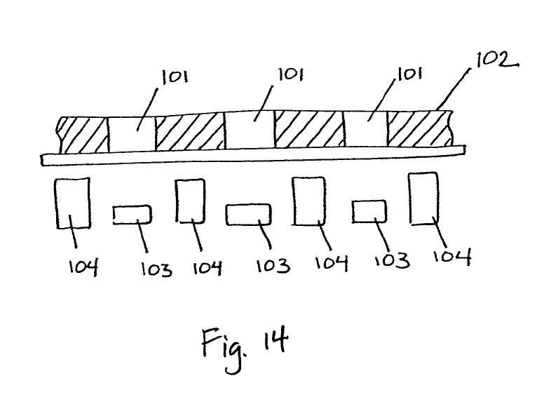

FIG. 13 depicts one embodiment of one or more storage cells being subjected to temperature control such as to promote a chemical reaction. Such chemical reaction may a reaction that is initiated or promoted by the application of heat. Alternately, more complex chemical reactions may be achieved, including those where temperature control is needed, optionally including maintaining one or more controlled temperatures for one or more periods of time.

A particular type of chemical reaction covered by the present invention is the polymerase chain reaction (PCR) conducted in cells per this invention where lengths of nucleic acid are amplified by temperature cycling between two or more temperatures with application of nucleic acid primers, a polymerase enzyme and other chemical and biological reagents as is known to those skilled in the art. Similarly, the ligase chain reaction (LCR), as is known to those skilled in the art, is also explicitly covered by the present invention.

Per FIG. 13, one or more storage cells 91 optionally fabricated as part of a substrate 92 are thermally coupled to a temperature-controlled device 93. Said thermal coupling may arise by juxtaposition, or by actual physical contact. The effectiveness of said thermal coupling may optionally be improved by an intermediate layer 94 of substance having good thermal transfer characteristics, such as an appropriate grease or other solid material, by an appropriate liquid or by a gas such as helium, or by other technique known to those skilled in the art.

Device 93 is temperature controlled such that thermal coupling to cells 91 results in said cells also being temperature controlled at the same temperature as device 93 or at a similar or related temperature. The temperature of device 93 may be controlled, and adjusted upwards or downwards, optionally at controlled rates, by standard techniques well known to those skilled in the art as are generally available in a variety of commercially available thermocyclers.

If a temperature difference, measured by any known technique, exists between cells 91 and block 93, said difference may optionally be calculated and corrected for as a correcting offset applied to the temperatures of block 93 to achieve the required temperature at cells 91. Advantageously, such differences may be measured at two or more temperatures over a range of temperatures with correcting offsets being applied at any point over the range by linear or higher-order interpolation, with such corrections and interpolations being provided such as by a microprocessor based controller. Alternately, or in addition, two or more such cycles of temperature correction may be applied until temperatures attained by cells 91 are closer or acceptably close to that required.

Although FIG. 13 implies that the device 93 lies parallel with one side of substrate 92 containing storage cells 91, other arrangements are also covered. Thus, the substrate could be sandwiched between two such temperature-controlled devices. Alternately, multiple such devices could be positioned around a substrate of different, complex or irregular dimensions. Alternately, one or more temperature-controlled devices may be shaped such as to efficiently physically accept a sample-containing substrate of matching shape and/or size.

In addition, although FIG. 13 implies closed sample cells, other configurations are covered, such as cells open at one side, or doubly open at opposing sides, or of irregular shape and sample access, together with other configurations as are used for chemical reactions by those skilled in the art.

### EXAMPLE 15

### Temperature Controlled Reactions

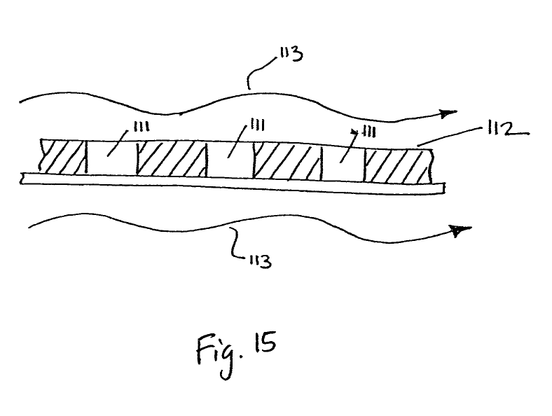

Alternative temperature control embodiments to Example 14 are possible. Per FIG. 14, one or more storage cells 101 optionally formed into substrate 102 may be temperature controlled by radiation heating from one or more sources 103 optionally with cooling to radiation sinks 104 or to the general environment.

To raise the temperature of cells 101, or to maintain an elevated temperature, radiated heat is applied from sources 103.

For cooling, or to achieve and maintain a lower temperature, heating from sources 103 or elsewhere is turned off or thermally masked, and cells 101 are allowed to lose heat by radiation to lower-temperature radiation sinks 104. optionally, sources 103 and sinks 104 may be combined into devices whose temperature may be controlled over the required range of temperatures.

Optionally, the temperature of cells 101 may be measured by a variety of means such as a directly connected or juxtaposed temperature sensor such as a thermometer or thermocouple or other temperature sensitive electrical device. Alternately, indirect measurements may be made such as by measurement of the radiation emission profile of cells 101 and/or of substrate 102, by measuring temperature sensitive coloration, or by other spectrometric techniques. All such techniques are well established and known to those skilled in the art. Advantageously, temperature errors (discrepancies between set and actual temperatures) may be measured and corrected for as outlined above. To assist in speeding temperature changes, the heating and cooling means 103 and 104 may optionally be set to a temperature significantly hotter or colder than that to which cells 101 are to be heated or cooled respectively, for a time calculated or empirically determined to be sufficient to cause most of the accelerated temperature transition to have occurred, at which point the temperature of means 103 and/or 104 may be adjusted to the actual temperature requested.

Advantageously, substrate 102 and/or cells 101 may be completely or partially coated with a surface coating with good heat radiation and adsorption properties, such as a black matt coating or similar.

In addition to the configuration implied by FIG. 14, also covered are configurations where heating and/or cooling sources are arranged in other configurations around cell and substrate assemblies of other arrangements and shapes, and also where storage cells may be of other shapes and closed or open.

### EXAMPLE 16

### Temperature Controlled Reactions

Further alternative temperature control embodiments to the above are possible. Per FIG. 15, one or more cells 111 optionally formed into substrate 112 may be temperature controlled by convection heating or cooling from convection current 113. Absent any alternative or interfering temperature control mechanism, the temperature of cells 111 will asymptotically approach or track that of convection current 113, and, after sufficient time, will adequately approximate to the convection current temperature. To assist in speeding temperature changes, the convection current 113 may optionally be set to a temperature significantly hotter or colder than that to which cells 111 are to be heated or cooled respectively, for a time calculated or empirically determined to be sufficient to cause most of the accelerated temperature transition to have occurred, at which point the convection current temperature may be adjusted to the actual temperature requested. As per Example 14, a number of established techniques are available for measuring the temperature of cells 111.

In addition to the configuration implied by FIG. 15, other physical configurations are covered, including the storage cells and substrate being in other shapes and form factors, convection currents being directed in other directions and orientations and storage cells being closed and open.

Detection Schemes

### EXAMPLE 17

### Fixed Direct Detection

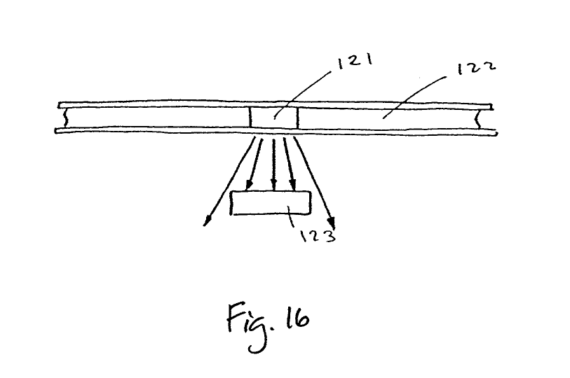

FIG. 16 indicates how a signal from a cell may be detected by juxtaposition or propinquity to a detection means.

The storage cell 121 optionally formed into substrate 122 may contain contents of which one or more components generate an optical signal or signals. Such optical signals may be generated by such as a chemiluminescence reaction. A detection means 123 is positioned such that the optical signal or signals impinge upon this detector and are detected and optionally measured or quantitated. Such optical detection means may be a photomultiplier, photodiode or photodiode array, charge coupled device or array, television camera, light sensitive film or any other known technique for qualitatively or quantitatively detecting light or an optical signal, as are known to those skilled in the art.

As an alternative embodiment, the contents of cell 121 may generate one or more radioactive signals, and detection means 123 is a radioactivity detector. Such radioactivity detection means may be a film, a geiger counter a scintillation counter or any other radioactivity detector as known to those skilled in the art. With this detector, the signal may be detected and optionally measured.

### EXAMPLE 18

### Parallel Direct Detection

FIG. 17 indicates how more than one signal from more than one cell may be detected by more than one detector.

Storage cells 131 optionally formed into substrate 132 are positioned such that each is juxtaposed to a detector 133. Contents of a cell may emit an optical signal in which case the detector may be an optical detector, or may emit a radioactive signal in which case the detector may be a radioactivity detector. The signals from one or more cells may be detected and optionally measured simultaneously through the matching detectors. The depicted optional masking element or spatial filter 134 may usefully obstruct the optical or radioactive energy emitted from a particular cell from reaching any detector other than the intended or juxtaposed detector, thereby usefully eliminating or reducing ‘cross-talk’ between one cell-detector pair and one or more other such pairs.

### EXAMPLE 19

### Optical Detection by Absorption Spectroscopy.

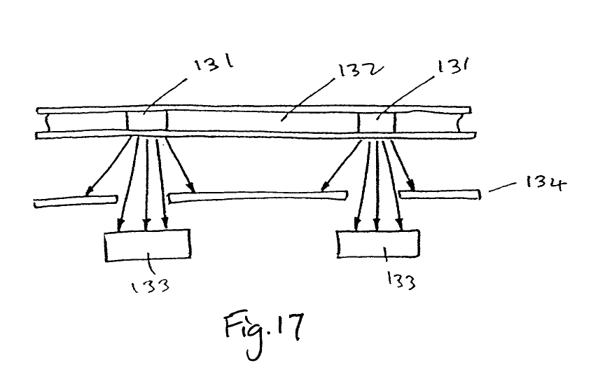

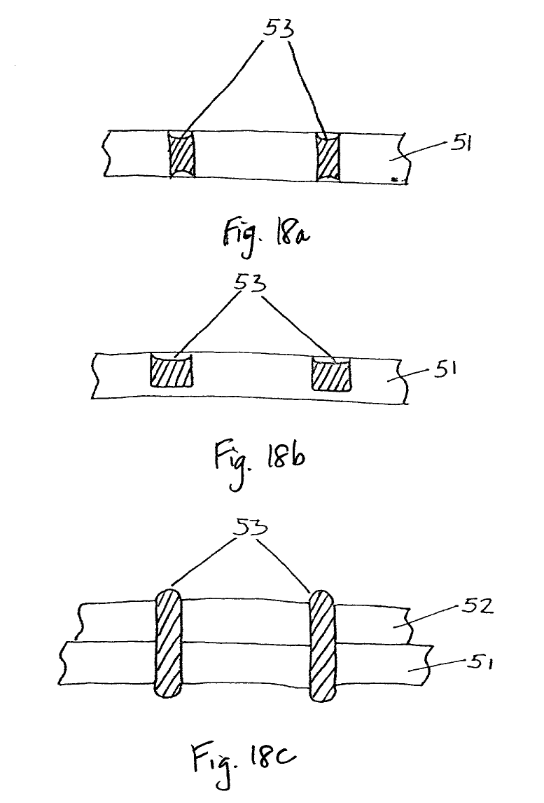

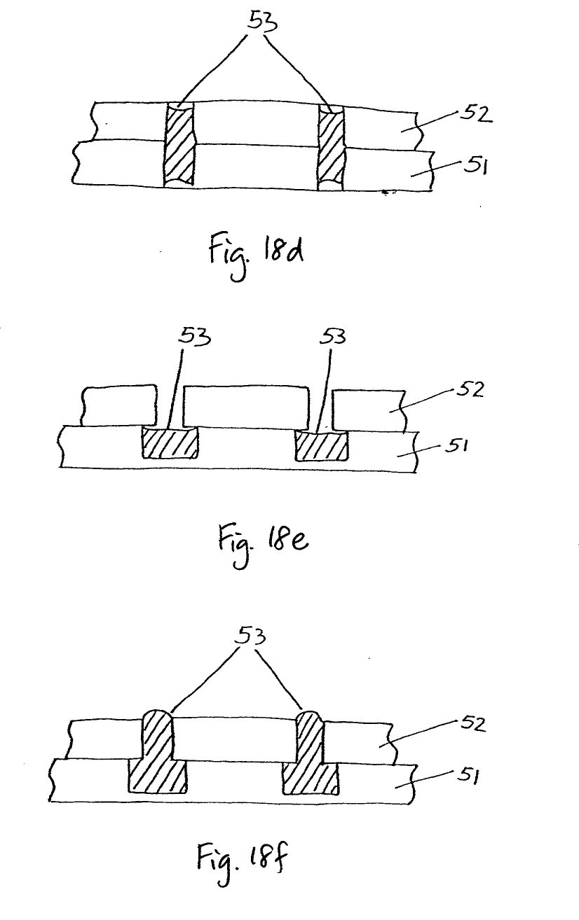

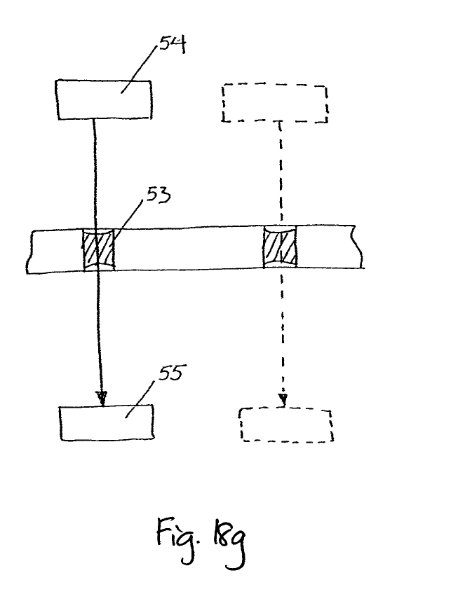

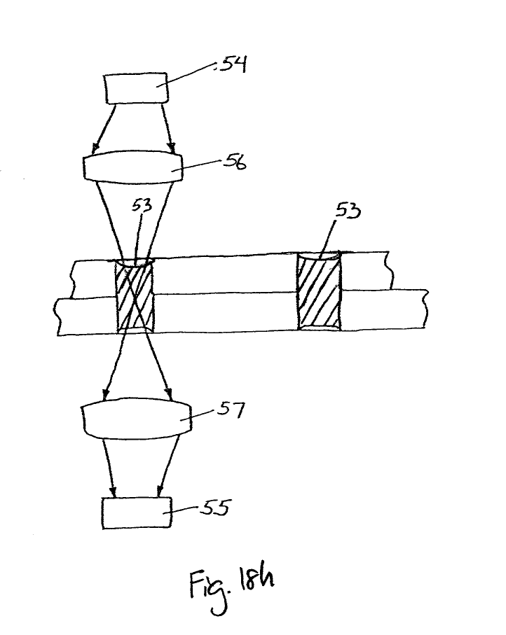

FIGS. 18a -18f depict various configurations of storage and assay sheet 51, either used singly or as a combination with stacked sheets 52 as described above, with various combinations depicted of storage cells 53 (both unitary and stacked) underfilled or overfilled with fluid optionally containing dissolved material, where one or more (not shown) sheets 52 may be stacked on sheet 51. One or more unitary or stacked cells 53 are positioned to be optically accessible. Per FIG. 18g, optical energy is caused to pass through cell 53 from a source 54 to a detector 55. If any cell has a closed bottom, said cell may be made from material transparent to the optical energy being used. Detector 55 measures optical energy passing through the cell 53, from which the optical absorption of the cell and contents may be determined.

Advantageously, the optical system may embody at least one monochromator (not shown) to distinguish the different wavelengths of optical energy, with most advantageously a scanning monochromator being used to measure the absorption spectrum of the cell and its contents such that the absorbance of sheet material and other cell contents may be distinguished from the optical spectra of one or more chemical species within the cell.

Per FIG. 18h, a lens 56 or more complex optical system may be used to focus or concentrate optical energy from source 54 onto cell 53 and its contents as indicated or by other optical configuration. Additionally, a lens 57 or other optical system may be used to focus or concentrate or focus optical energy emerging from the cell onto detector 55.

Per FIG. 18i, a mirror 59 or more complex optical system may be used to focus or concentrate optical energy from source 54 onto cell 53 and its contents as indicated or by other optical configuration. Additionally, a mirror 59 or other optical system may be used to focus or concentrate or focus optical energy emerging from the cell onto detector 55.

Additionally, other lenses, mirrors or mixed combinations, or other optical systems (not shown) as will be known to those skilled in the art may be used to increase the optical efficiency and/or sensitivity of the detection means.

### EXAMPLE 20

### Optical Detection of Fluorescence or Luminescence.

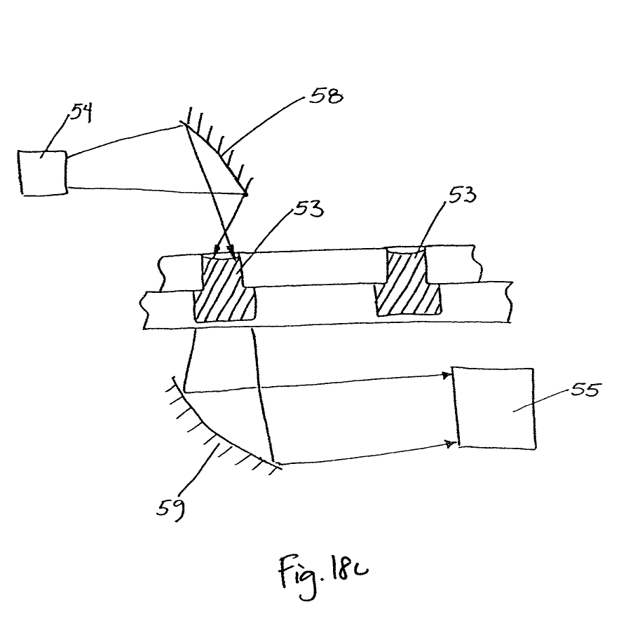

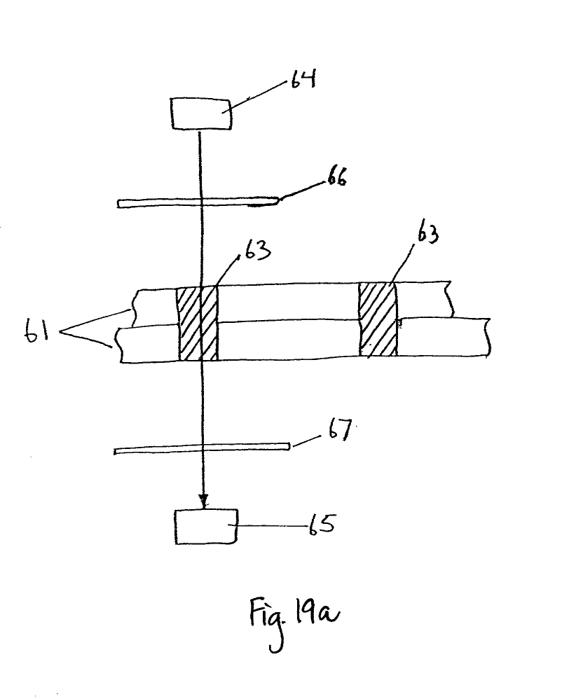

FIG. 19a depicts a sheet, or combination of stacked sheets as described above 61, placed such that one or more cells 63 therein are optically accessible. Source 64 irradiates cell 63 with optical energy causing fluorescence of one or more substances located within cell 63. Optionally, filter 66 may be used to remove or attenuate optical energy at wavelengths other than causing fluorescence. Detector 65 detects and optionally measures fluorescence from one or more substances within cell 63. Optionally, filter 67 may be used to remove or attenuate optical energy at wavelengths other than that to be detected as fluorescence. Advantageously, either or both such filters may be replaced by monochromators better able to delimit excitation and fluorescence wavelengths. Preferably, one or more monochromators may be scanned, such that one or more fluorescence spectra may be obtained to better detect the fluorescence signals from one or more fluorescing substances and to distinguish said signals from each other and from background fluorescence and thereby to measure same.

Per FIG. 19b, lenses 68 and 69 may be used to focus or concentrate optical energy from source 64 onto cell 63 and thence to detector 65 with one or more optical filters 66 and 67, or alternately one or more monochromators being used as above to assist with measuring and interpreting the fluorescence signals.

Per FIG. 19c, by an alternate optical configuration, source 64 irradiates cell 63 with excitation optical energy, optionally through filter or monochromator 66, and fluorescence optical energy reaches detector 65 through lens or other optical system 69, optionally through filter or monochromator 67. Spatial filter 68 eliminates or reduces the amount of excitation energy reaching detector 65, advantageously assisting in distinguishing fluorescent from excitation energy. Alternately, other known dark field configurations may be utilized.

### EXAMPLE 21

### Optical Detection of Luminescence

Per FIG. 20, represents an optical system whereby optical energy from a cell 73 formed in one or more stacked sheets may be imaged, focused or concentrated onto detector 75 by one or more mirrors 76 and 77. Advantageously, ellipsoidal mirrors maybe utilized such they efficiently image a relatively small cell onto a similarly small cell compared with the dimensions of the overall optical system. An efficient optical system as indicated may provide virtually spherical capture of optical energy emitted by substances within cell 73. Alternate optical configurations may collect less of the optical energy, such as because required optical sensitivity so permits, or because mechanical/steric considerations block complete optical access. For example, optical energy may be collected from just one side of the sheet or stacked sheets, by the utilization of just a single mirror. Such an optical configuration may advantageously be used to capture, detect and optionally measure optical energy originating within cell 73 such as a luminescence or chemiluminescence signal. Alternately, one or more lenses may be used to capture the optical chemiluminescence signal by known means.

Alternately, such an optical system, or similar, may be used for detection of other optical signals. For example, a similar optical system may be used for detecting a fluorescence signal by conducting excitation optical energy to cell 73 such as by a hole (not shown) in one of the mirrors, or by other known means.

### EXAMPLE 22

### Mass Spectroscopic Detection of Chemical Species

Although not herein illustrated, chemical species or other material placed in storage cells, or stored therein, or participating in chemical reactions, or purification or concentration steps or processes therein per the current invention may be detected my mass spectroscopy (otherwise termed mass spectrometry).

In one example, such mass spectroscopic detection may be effected by liquid/fluid transfer by known means from one or more cells and ionization of materials therein by atmospheric chemical ionization (APCI) or electrospray ionization using known methods, as will be apparent to those skilled in the art.

Alternately, matrix assisted laser desorption and ionization (MALDI) may be effected by adsorbing material of interest onto known matrix material and then using laser power to desorb and ionize said material by standard means. To achieve this, said material may first by transferred by any known means to a device or substrate holding matrix from which such ionization may be induced. Alternately, matrix material may be located on sheet material per the current invention, particularly within any cell type or combination as herein described, if the overall analytical or other procedure so permits or is so facilitated, and, after material of interest has been adsorbed onto such matrix material, the laser assisted desorption and ionization may be induced directly from such sheet or cell by manipulation such as to interface same by any known means to the input of any type of mass spectrometer, but particularly advantageously a time of flight (TOF) system, such that one or items of that material within the sheet or cell may be so offered for analysis by any known mass spectroscopic analytical technique such as are known to those skilled in the art.

### EXAMPLE 23

### Scanned Direct Optical Detection

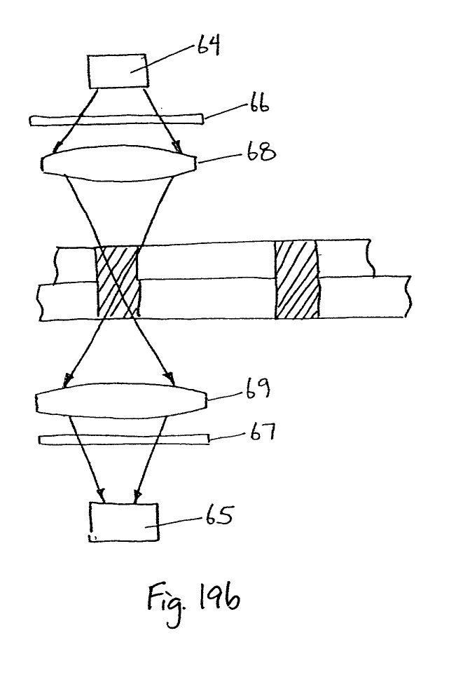

FIG. 21 indicates how one or more optical signals from one or more storage cells 141 may be detected sequentially by successively positioning them within the effective range of one or more detectors 143, with optional spatial filter or masking element 144 reducing cross-talk between adjacent detectors.

Covered configurations include where the cells are moved past one or more detection means, or where the detection means are moved with respect to the cells as illustrated, or where both cells and detection means are moved in such a way that a given cell may be detected by one or more detection means, or where several cells may be sequentially or in parallel detected by one or more detection means, or where all or part of an ensemble of cells may be detected by one or more detection means.

### EXAMPLE 24

### Detection by Capture or Imaging of Optical Signal

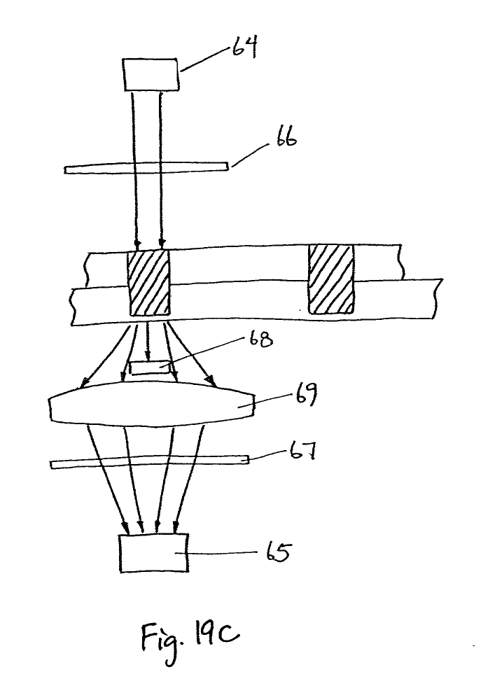

FIG. 22a depicts the use of an optical element such as a lens 153 to couple an optical signal from a storage cell 151 optionally formed into substrate 152, to an optical detector 154.

Although a simple convex lens is depicted, more complex optical formulations could be used. Alternatively, one or more mirrors could be used to couple the optical signal from the cell to the detector. In a simple embodiment, the optical element may be used simply to gather some amount of optical energy from the cell and direct this onto a detector. Alternately, an image of the cell may be formed on the detector.

For the optical energy to be quantitatively measured by the detector, per FIG. 22b, it may be advantageous for the cell's image to underfill the area of the detector 154 such that no energy misses the detector and is wasted and not measured. This may be particularly advantageous if, because of positioning uncertainties or errors in the cell, the optical element or the detector, or any combination of these, the exact positioning of the cell image on the detector is uncertain such that as the detectable image is smaller than the physical detector dimensions, and this underfilling usefully provides for some positioning error before the image leaves the detector. This may also be advantageous if there is any vibration in the system, to avoid the vibration-induced motion of the image on and off the detector modulating the detected signal amplitude.

Alternately, per FIG. 22c, the image may be arranged to overfill the detector 154, such that any relatively small positional error or vibration may keep the detector within the expanded optical image or energy envelope, with advantageous reduction of vibration-induced modulation.

### EXAMPLE 25

### Parallel Capture or Imaging of Optical Signals

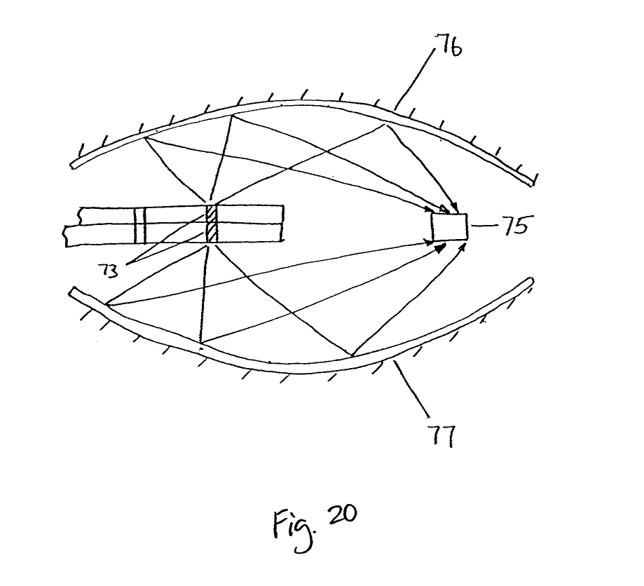

Per Example 24 above, a single optical element may image the optical signal from a single cell onto a single detector. More complex schemes may be constructed and are all included within the present invention. For example, per FIG. 23, a single optical element 164 may be used to simultaneously conduct or image the optical signals from two or more storage cells 161 and 162 optionally formed into substrate 163, onto two or more detectors 166 and 165 respectively, where the cells may be arranged in an ensemble or array with detectors advantageously arranged in a matching array.

As examples of this, the detector may be physically separate, or it may be an array detector such as CCD array or a television camera or equivalent, where an array of optical pixels is monitored and each detected signal may come from one or more pixels and multiple detected signals may be deduced from the individually detected and measured array pixels.

### EXAMPLE 26

### Capture or Imaging of Arrays of Optical Signals

Per FIG. 24, a single optical element or more complex optical scheme 175 may image the optical signals from an ensemble or array of cells 171, 172 and 173 onto a composite detector 176 or matching array of detectors. Masking elements or spatial filters (not shown) may be used to eliminate or reduce the possibility of optical energy from any cell impinging upon any detector other than that onto which it is intended to be directed or imaged.

Two or more cells may have their optical signals conducted to a single detector, where the optical signals may be detected and measured together, such as to determine whether at least one of a group of two or more cells is generating an optical signal, or to detect and measure optical signals additively with the option of mathematically separating such signals.

### EXAMPLE 27

### Detection by Scanning of Optical Signals

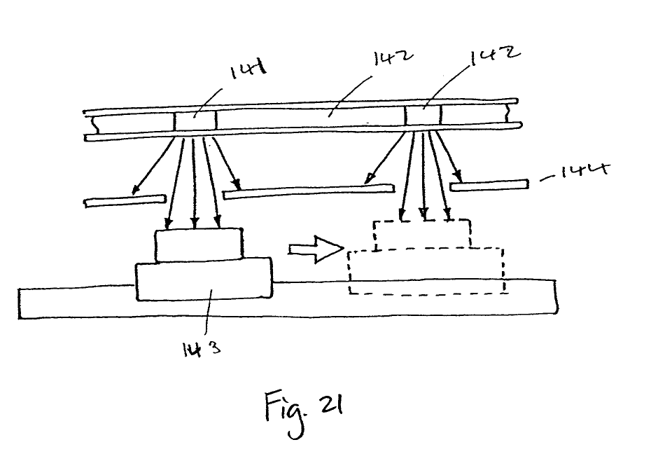

FIG. 25 depicts the use of an optical element 182 to scan optical signals from one or more cells 181 onto at least one linear optical detector 183, such as a linear CCD array. More than one cell, or more generally an ensemble or array of cells, may have their optical signals detected and optionally measured.

Although a simple cylindrical lens is depicted, a compound lens may be used, or one or more mirrors may be used to image optical energy emanating from the cells. As depicted, the lens simultaneously images the optical energy from more than one cell onto the linear detection array. Analysis of the optical signal detected along the length of the linear detection array may be used to infer the existence and optionally the magnitude of the optical signal from each cell imaged onto the detection array. One or more optional masking elements (not shown) may be used to eliminate or reduce the incidence onto the detector of optical energy from cells not being directly imaged onto the detector.

By motion of the ensemble of cells, or the lens or the detector array (or any appropriate combination) different combinations of cells may have their optical energy imaged onto the detector array. Therefore, by such means, all or part of a two-dimensional ensemble or array of cells may be sequentially imaged onto the linear detection array.

### EXAMPLE 28

### Capture or Imaging of Arrays of Optical Signals

Per FIG. 26, an optical means 192 (built from one or more lenses or mirrors or any combination thereof) may image the optical signals from a two-dimensional ensemble or array of cells 191 onto a detector array 193 such as a two-dimensional array of discrete detectors or onto a two-dimensional CCD array or TV camera or similar area sensitive optical detector.

If the field of view of the optical imaging system is sufficient, an entire ensemble or array of cell's optical signals may be simultaneously imaged onto the detector array, and the individual optical signals may be identified from their spatial positions on the detector array and may be detected and optionally measured. Alternately, a set of cell optical signals may be imaged onto the detector at one time for detection or measurement, following which the ensemble or array of cells or the optical element or the array detector (or any combination) may be moved such that a different set of cell signals is imaged onto the detector. By this means, an indefinite number of cells may be sequentially imaged onto the detector as one or more sequential sets of images.

### EXAMPLE 29

### Lens at Cell to Improve Detection of Optical Signals

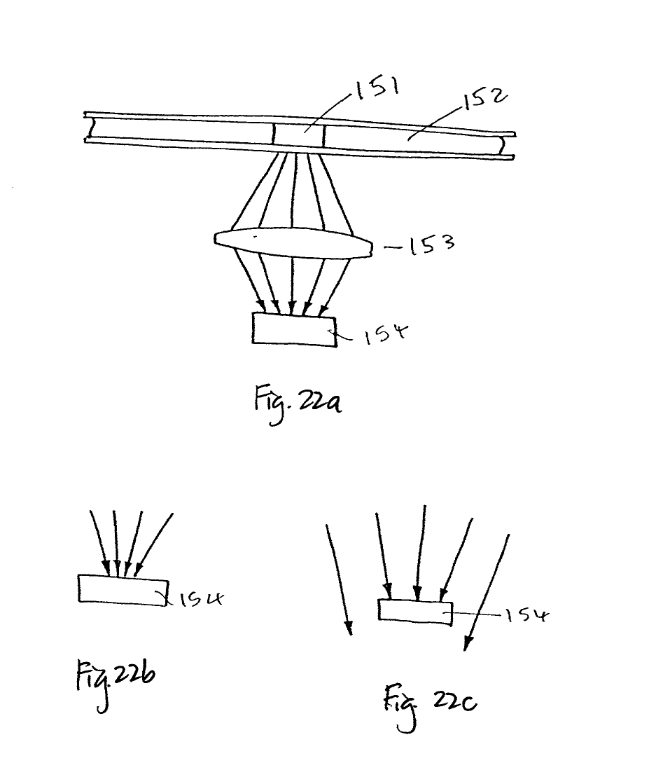

Per FIG. 27, a cell 201 from which an optical signal is to be detected may have a lens 202 formed in a one of the walls forming the cell. Alternatively, the lens may be mounted flush or close to a wall of the cell, such as by juxtaposition, welding or adhesion to the cell wall. Although the lower wall is depicted, any other wall of the cell may be used. Said lens 202 may advantageously be formed as a convex lens such that it focuses optical energy onto an external detector 203, or that it narrows an otherwise divergent beam of rays 204 such that more optical energy may be captured by a detector 203. Said lens 202 may function by itself or it may form part of a compound optical system. In one preferred embodiment, the lens 202 may be formed from clear plastic. Alternately, the lens may be formed from a clear fluid contained within an envelope. Alternatively, all or part of the optical arrangement may be provided by a fresnel lens formed in the sidewall off the cell or of a juxtaposed means. Other embodiments are possible, as will be apparent to those skilled in the art, and all such alternative embodiments are included within the present invention.

### EXAMPLE 30

### Filter at Cell to Improve Detection of Optical Signals

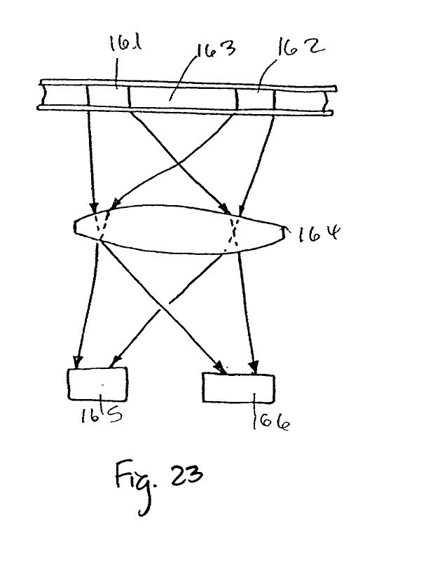

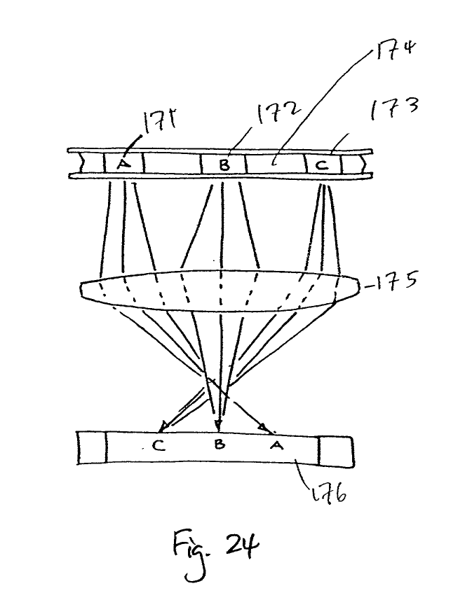

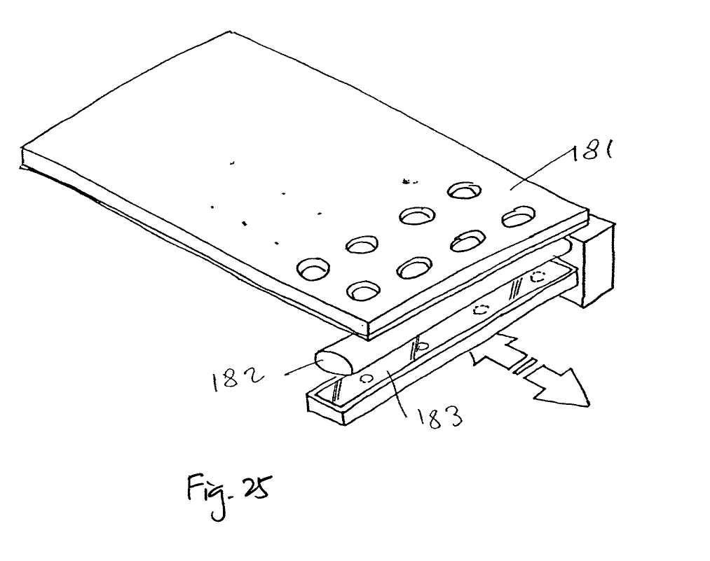

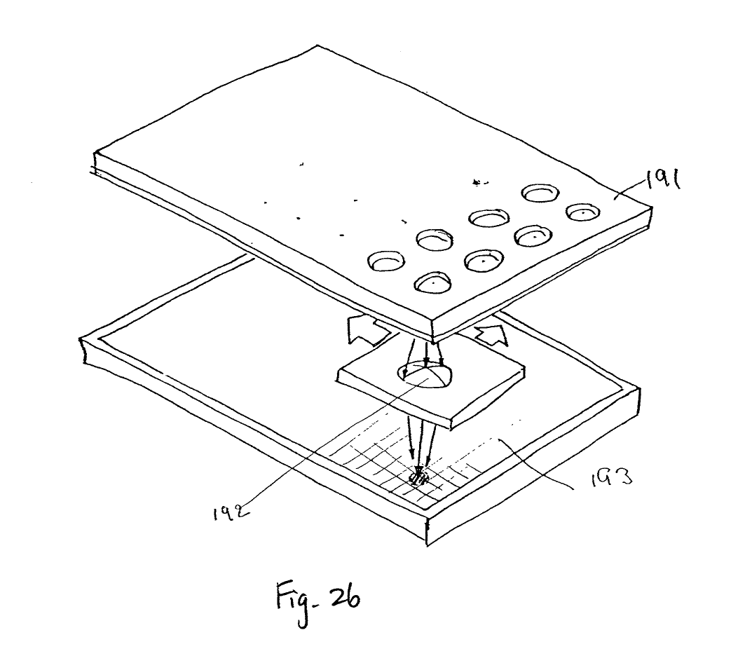

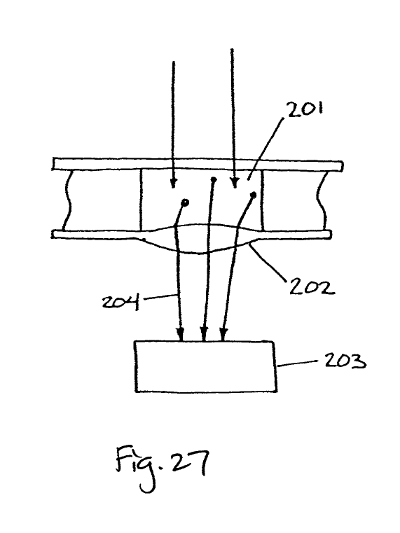

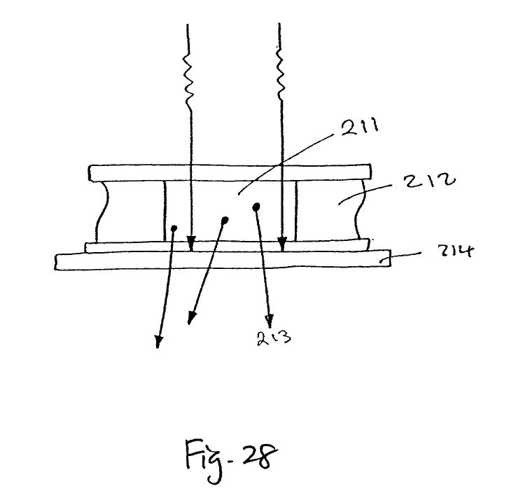

Per FIG. 28, a cell 211 optionally formed in substrate 212 from which an optical signal 213 is to be detected may have a filter 214 formed in a one of the walls forming the cell. Alternatively, the filter 214 may be mounted flush or close to a wall of the cell, such as by juxtaposition, welding or adhesion to the cell wall. Although the lower wall is depicted, any other wall of the cell may be used. One purpose of the filter may be to limit the wavelengths of optical energy allowed to reach an external detector (not shown). This scheme may be advantageously used with fluorescence detection where the filter may preferentially reduce the level of excitation energy reaching the detector while fluorescence optical energy is preferentially transferred to the optical detector.

Alternately, the filter may be used to limit or modify the optical energy applied to the cell. Although not shown, two or more filters may be attached to a cell to simultaneously filter the applied and detected optical energy, in any combination. In a preferred embodiment, said filters may be formed from plastic or glass with appropriate optical properties. One or more layers of material, optionally with differing optical or other properties, may be used to form the filter layers. The optical properties may be derived from selective optical absorption, or from dichroic interference, or other filtering techniques, or any combination of known techniques. Other embodiments are possible, as will be apparent to those skilled in the art, and all such alternative embodiments are included within the present invention.

### EXAMPLE 31

### Reflective Layer at Cell to Improve Detection of Optical Signals

Per FIG. 29a, a cell 221 optionally formed within substrate 222 from which an optical signal is to be detected may have a reflective layer 223 formed in or at one or more of the walls forming the cell. Alternatively, the reflective layer may be mounted flush or close to a wall of the cell, such as by juxtaposition, welding or adhesion to the cell wall. Although the upper wall is depicted, any other wall or region of the wall or container of the cell may be used. The reflective layer may be of a single material or it may be a multilayer formulation. The materials may include metallic material such as aluminum foil or other metal, or dichroically reflecting combinations of layers, together with plastic or glass or combinations thereof. One purpose of the reflective layer may be to restrain optical energy from escaping from the cell and reflecting this back into the cell to assist with an optically induced chemical reaction. An alternative approach may be to reflect optical energy back through the cell for detection elsewhere—such as through the opposite wall of the cell. FIG. 29(a) depicts a simple case of a basically flat reflective layer that is formed or positioned at the wall of the cell.

FIG. 29b depicts the case where the reflective layer 223 is shaped, such as by being formed over a spacing element 224 (which may external to, or be thickening or equivalent, of the cell wall).

Alternatively, FIG. 29c depicts the case where the reflective layer 225 is shaped by being formed over, or matched to, the shaping of the cell wall 226. With cases (b) and (c), the curvature may optionally be arranged to have useful optical qualities such as serving as a concave mirror which may direct energy to or generate a focused image on a detector, or may direct or focus optical energy within the cell or towards a particular region within the cell.

### EXAMPLE 32

### Measuring Optical Absorption of Cell and Contents

Per FIG. 30, a cell may be configured as part of an optical system such that optical properties of the contents of the call may be measured. Incident energy 231 is applied to the cell 232 and, after passing through all or part of the cell, this energy is measured by an optical detector 233. One form of such optical measurement may be optical absorption, by measurement of the ratio of applied and emerging energy and calculation of that absorbed by or within the cell. Such optical absorption measurement may be conducted in the ultraviolet, visible or infrared regions, by techniques understood by those skilled in the art.

Alternatively, the optical measurements could be made of optical polarization rotation, light scattering, turbidity, or other optical property or combination etc. Of particular utility may be optical fluorescence measurement with its superior sensitivity, whereby optical excitation energy of a shorter wavelength is applied to the cell contents and fluorescent energy of a lower wavelength may be generated and detected.

### EXAMPLE 33

### Additional Optical Elements to Optical Detection

Per FIG. 31, Example 32 may be improved by additional optical elements such as one or more selective filters 241 applied to improve the optical efficiency or simplicity of the overall optical system. Thus, as a specific case, by the use of one or more appropriately narrow optical bandpass filters, a specific optical absorption measurement may be able to be accomplished with acceptable and useful accuracy without recourse to potentially expensive monochromators. Alternatively, a fluorescence measurement may be made if filter 241 sufficiently attenuates the applied excitation energy reaching the detector.

### EXAMPLE 34

### Blocked Excitation for Fluorescence Measurements

Per FIG. 32, a cell 251 may be configured as part of an optical system performing fluorescence measurements of the cell contents, with darkfield illumination. In this example, a spatial filter 252 is configured to block the excitation energy 253 applied to the cell from directly reaching detector 254. Fluorescent energy that is emitted more isotropically is gathered by the depicted lens 253 (or, in general, an optical system) and focused or directed onto the detector 254. The spatial filter 252 prevents the directly transmitted excitation energy from reaching the detector.

### EXAMPLE 35

### Direct Fluorescence Measurements

FIG. 33 depicts a direct fluorescence measurement. Cell 261 optionally formed in substrate 262 contains contents that may have fluorescent properties. Incident energy 263 is applied through filter 264 which passes the excitation energy, but eliminates or attenuates energy present at the detection wavelength. Fluorescent energy 265 is passed to detector 267 through filter 266 which passes the detection wavelength but eliminates or attenuates the excitation energy wavelength. Both depicted filters may optionally be formed from multiple actual filters combined so as to provide the required optical properties.

### EXAMPLE 36

### Blocked Excitation for Fluorescence Measurements

FIG. 34 represents an example of optical fluorescence detection where the configuration is arranged such that the excitation energy does not directly reach the detector. In this configuration, the excitation energy 271 is directed as a beam into the cell 272 by means of a mirror 273, with the arrangement that the same mirror directs away from the detector 275 any excitation energy directly returned (such as by fresnel reflection) from the cell. The lens 274, or other optical system, captures fluorescence energy from a wider solid angle than that intersected by the mirror. Advantageously, a filter 276 may be used as indicated to attenuate excitation energy that will otherwise indirectly reach the detector. Advantageously, this positions all optical elements on one side of the cell.

### EXAMPLE 37

### Fluorescence Detection with Darkfield Excitation

Per FIG. 35, a cell may be configured within an optical system for fluorescence detection with darkfield excitation. In the scheme depicted, optical excitation 282 is applied from a relatively wide solid angle by lens 283, whereas the fluorescence signal is gathered for detection from a narrower solid angle within which excitation energy, which would otherwise reach the detector, has been blocked by spatial filter 284. Optionally, spatial filter 286 may be used to specifically block excitation energy that leaves cell 281 and may thereby generally improve the optical system's stray light performance.

### EXAMPLE 38

### Fluorescence Detection with Darkfield Excitation

FIG. 36 shows an alternative optical configuration whereby a cell may be positioned for fluorescence detection with darkfield excitation. In the scheme depicted, optical excitation is applied from a relatively narrow solid angle 292 to cell 293, whereas the fluorescence signal is gathered for detection from a wider solid angle 297 by lens or optical system 296. Spatial filter 294 prevents excitation energy from directly reaching the detector 298.

As a refinement to the above, optional mirrors 299 may be used to reflect back into cell 293 fluorescence signal that would otherwise be lost, such that this can emerge from the cell within the solid angle capture envelope of the detection optical system 296. Similar mirrors could be used also with Example 37 above.

### EXAMPLE 39

### Fiber Optic Signal Detection

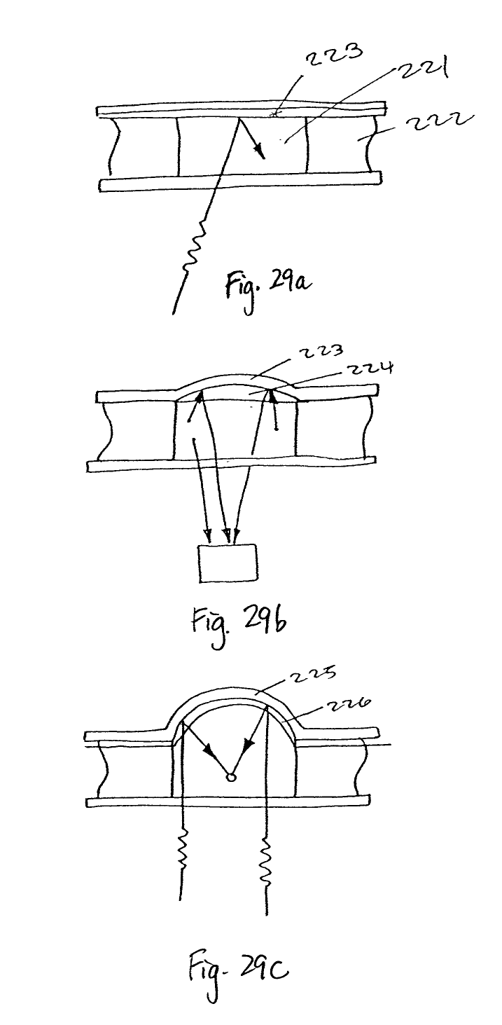

FIGS. 37a and 37b depict use of fiber optics to facilitate optical detection. Per FIG. 37a, one or more cells 301 optionally formed in substrate 302 are positioned such that emitted optical energy is routed into one or more optical fibers 303 optionally formed in fixture 306, and delivered to one or optical detectors 304. Additional optical elements (not shown) such as lenses, mirrors or combinations may advantageously direct optical energy into and out of the optical fibers with improved efficiency and resultant detection sensitivity. Alternatively a single detector may be used sequentially positioned at the end of one or more optical fibers, with cost economies and invariant sensitivity.

Per FIG. 37b, to capture energy from an array of cells, optical sheets 306 may be stacked as shown to give a two-dimensional detection capability. This may be provided with a two-dimensional area detector covering the array of optical fiber outputs in parallel or positioned to detect the entirety in subsets. Alternately, a linear detector array may be consecutively positioned to detect linear subsets of the array of optical fiber outputs. Alternately, a single detector may be positioned to detect one or any combination of the optical fiber outputs.

### EXAMPLE 40

### Electrochemical Detection

FIG. 38 depicts one or more cells 311 optionally formed into substrate 312 with two or more electrodes 313 accessing the fluid interior of the cell. Electrochemical measurements are used to detect one or more chemical constituents within the cell, to measure the concentration of such constituents, to detect that one or more chemical reactions have occurred, and/or to detect or measure the products of such chemical reactions.

Such measurements may be made potentiometrically (voltametrically) by measuring an electrical voltage representing one or more chemical entities. Alternatively, measurements may be made amperometrically by measuring an electric current representing one or more chemical entities. One or more membranes may be optionally employed to aid with specificity of the chemical species being monitored. Chemical mediation may optionally be employed to make the reaction chemistry accessible by electrochemical means. All such techniques and combinations thereof are well known to those skilled in the art.

Devices with Exterior Transfer Channels

### EXAMPLE 41

### Forward Motion Quill

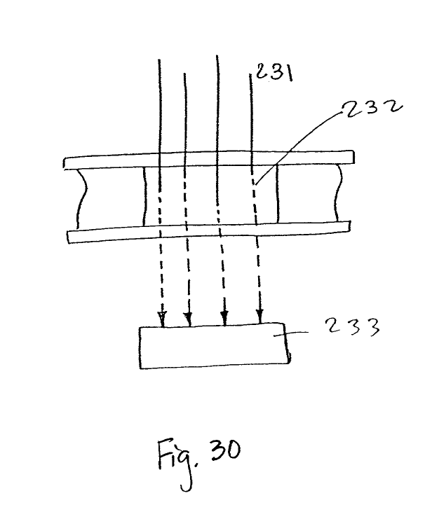

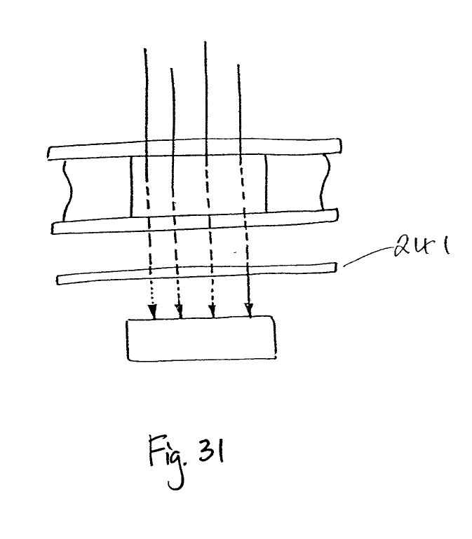

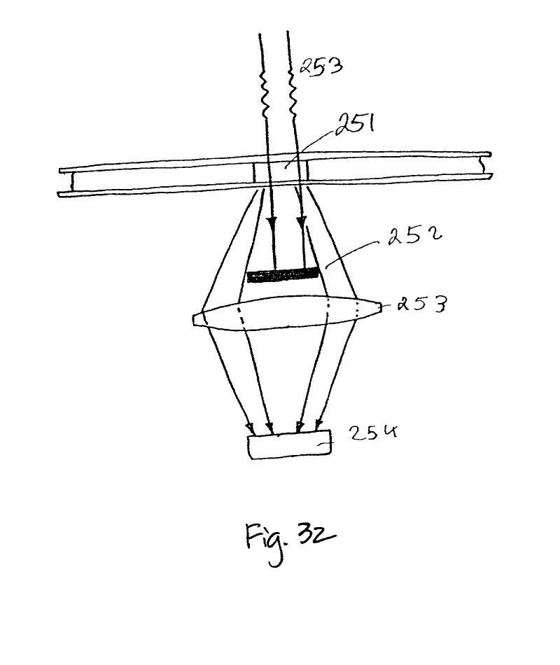

FIG. 39a provides a cross-sectional view of a single miniature quill device, shown in its nominal content storage position. This quill is preferably molded in polypropylene or other polymeric material resistant to solvents and biological reagents, or may be fabricated of stainless steel or other appropriate material. Spring-loaded quill 11 within storage reservoir 12 is sealed by annular seal 13 and by reverse seal 14 both of which may be separate elements or formed in the reservoir, such as shown. An optional spring may assist with quill positioning.

As shown in FIG. 39b, when quill 11 is depressed, leak path 15 forms and allows fluid to flow into one or more grooves or slits 16 by gravity and/or capillary action, optionally assisted by hydrostatic pressure applied from above. Slits 16 lead to quill tip 17, where the fluid forms a drop, available for transfer. The amount transferred to tip 17 is based on the geometry of slits 16 and seal 15, the rapidity and distance of quill 11 movement, fluid viscosity, the geometry of tip 17 and the surface tension of the fluid thereto, and applied pressure if any.

As shown in FIG. 39c, quill tip 17 may then be brought into contact with a solid or fluid surface 18, which draws the fluid from the quill by gravity and/or surface tension, or by induced flow e.g. by pressure, or by any combination. Surface 18 may be a flat or contoured surface, a cavity or well, or storage device. Tip 17 may optionally be retracted such as to a nominal storage/closed position.

As indicated by FIG. 39d, the volume of fluid may separate from tip 17 such that a quantity of fluid has then been dispensed from reservoir 12 to surface 18. A variety of tip shapes 17 may optionally be employed such that the propensity to retain fluid, or the volume of fluid that may be retained may advantageously be varied or controlled, as may the ability for a fluid drop to cleanly separate. The dispensing a quantity of liquid may optionally be repeated such that one or more further quantities are dispensed at the same location, or by movement of the reservoir assembly or of the surface, or both, or to dispense one or more quantities of fluid at different locations. Optionally, continuous flow may be achieved, such as for a timed period, or until a particular volume has been dispensed. Tip 17 may, before or after dispensing the fluid, be rinsed such as by being dipped into a surface of another liquid (not shown) or by being irrigated by an applied flow of an other liquid (not shown), for cleaning purposes.

### EXAMPLE 42

### Reverse Motion Quill

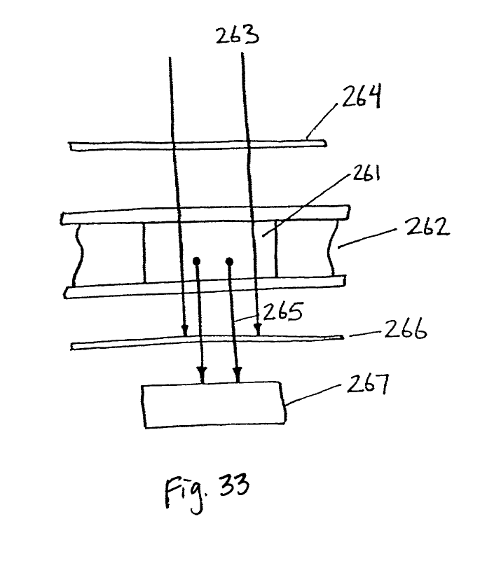

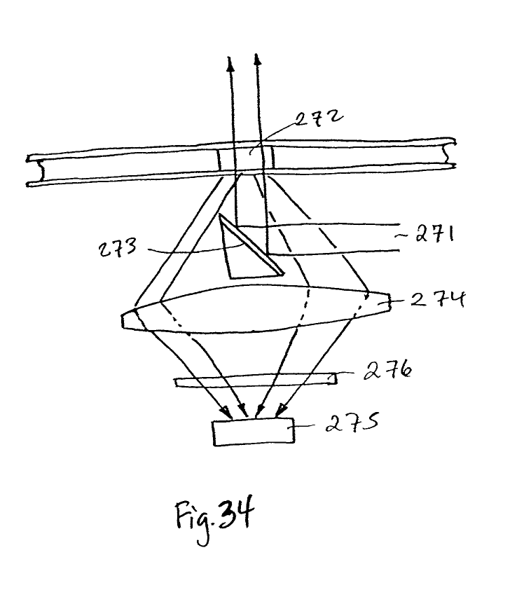

FIGS. 40a-d depict an alternate embodiment that uses two annular seals. This arrangement provides a more positive seal at the bottom and may eliminate the need for optional spring-loading.

Per FIG. 40a, needle 21 (which optionally may be a rod, probe or a shaft) is encapsulated by storage reservoir 22 and is sealed by top seal 23 and bottom seal 24, either of which may be formed in the reservoir or may be a supplied as a separate element. As an option, the, lower internal contour of reservoir 22 may be shaped such as to catch sediment so that this does not enter nor contaminate the seal nor be dispensed with the fluid.

Per FIG. 40b, when needle 21 is raised, leak path 25 develops, allowing fluid to flow into one or more grooves or slits 26 by gravity and/or capillary action, and/or applied fluid pressure. Slit or slits 26 lead to needle tip 27, where the fluid forms a drop, ready for transfer. The drop amount available for transfer is based on the geometry of slit or slits 26, the rapidity and distance of the needle 21 movement, the geometry of tip 27, fluid viscosity, the surface tension of the fluid thereto and optionally applied hydrostatic pressure.

Per FIG. 40c, needle 21 is then lowered, sealing off transferred fluid from that in reservoir 22. Needle tip 27 is brought into contact with a solid or fluid surface 28, to which fluid flows from the quill by gravity and/or surface tension.

Per FIG. 40d, needle tip 27 is raised from the solid or fluid surface 28, such that a quantity of liquid has effectively been transferred from reservoir 22 to solid or fluid 28. A single quantity of fluid may be so dispensed, controlled for example by the drop size. Multiple such drops may be deposited at the same or different locations. Alternately, fluid may be transferred on a continuous flow basis, where the amount transferred will be defined by the average flow rate and time of flow, either or both of which parameters may optionally be controlled.

Multiple devices may be placed in an ensemble, such as a linear or two-dimensional array, to optionally achieve multiple or parallel actuation.

Alternately, an array of transfer needles could be separate from the reservoirs, and arranged to access sealed reservoirs by piercing a membrane. The array of needles can optionally be fabricated as one disposable plastic part for ease of manufacture and/or low cost.

### EXAMPLE 43

### Quill Transfer with Fluid Assistance

FIG. 41 shows another embodiment of this device with a hollow needle 31 designed to allow the addition of additional liquid, such as diluent, buffer or reagent solution, during the overall fluid dispense cycle. Liquid flow through e.g. needle 32 may be used to wash the dispensed fluid from the tip onto a surface 34 such as, for example, in an assay well, surface, well, reservoir or other container. The volume of additional liquid may advantageously be larger than that of dispensed fluid, so that the combined drop is larger and easier to release. The operating sequence for a device per FIG. 41 is similar to that shown in FIGS. 419a-d, except that the transfer sequence advantageously may not require contact with the target surface. Needle 31 may be hollow, enabling the second liquid 32 to rinse the dispensed fluid into the target surface. The resulting drop 33 or liquid stream may be much larger than the dispensed fluid volume, so that it may overcome surface tension and be released solely through gravity. Alternately, 32 may be applied air or gas that may be used to assist in transferring fluid and/or clearing residual fluid from the needle tip at any time.

Such devices advantageously may be fabricated in ensembles or arrays.

Meniscus Dispensing Device

### EXAMPLE 44

### Meniscus Dispenser

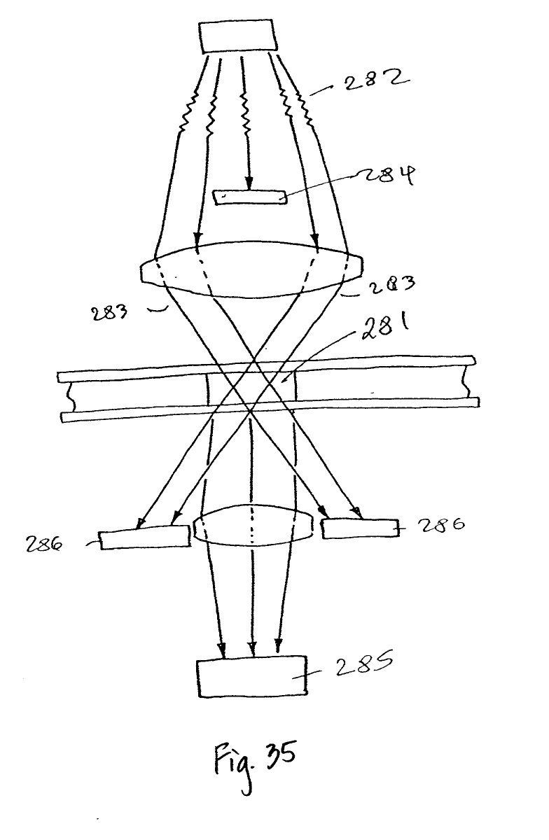

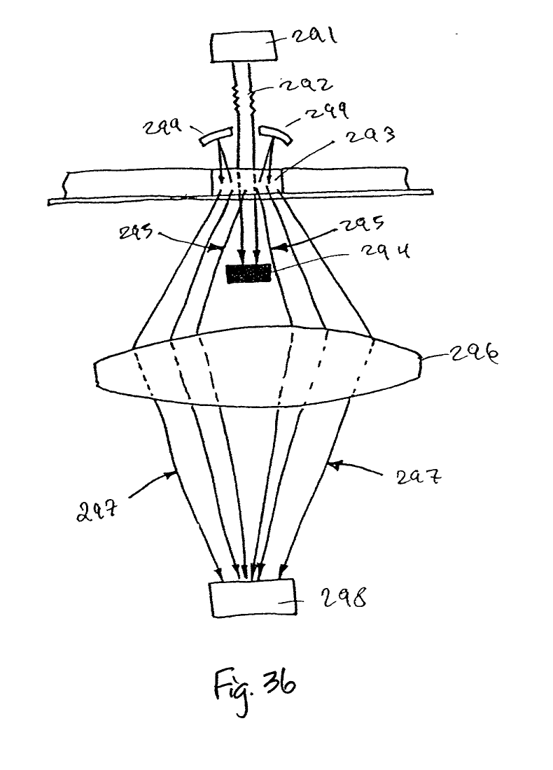

FIG. 42a depicts a cross-sectional view of a dispensing device, shown in its nominal storage position. Fluid is stored in reservoir 41, with sliding seal 42 positioned such as at the bottom and optionally vent 43 at the top. Sliding seal 42 covers fluid channel 44. Sliding seal 42 and vent 43 may be linked and move together, or may move separately.

In FIG. 42b, fluid channel 44 is open, allowing fluid to pass through and collect in open well 45. This well 45 has no bottom, but with appropriate fluid volumes and device geometries, surface tension will hold the fluid in place. Optionally, droplet formation may be assisted by tip 48 in the path of the fluid.

In FIG. 42c, sliding seal 42, vent 43 and nozzle 46 reach the end of their downward motion. Nozzle 46 now seals reservoir 41 and prevents further flow. A burst of gas 47, such as nitrogen, argon or air, then expels the fluid from the bottom of well 45 into surface or receptacle 49. Alternately, a flow of additional liquid may be used instead, such as water, diluent, buffer or reagent solution, to expel the fluid and optionally wash or clean the device.

### EXAMPLE 45

### Capillary Transfer Device for Storage, Metering and Dispensing

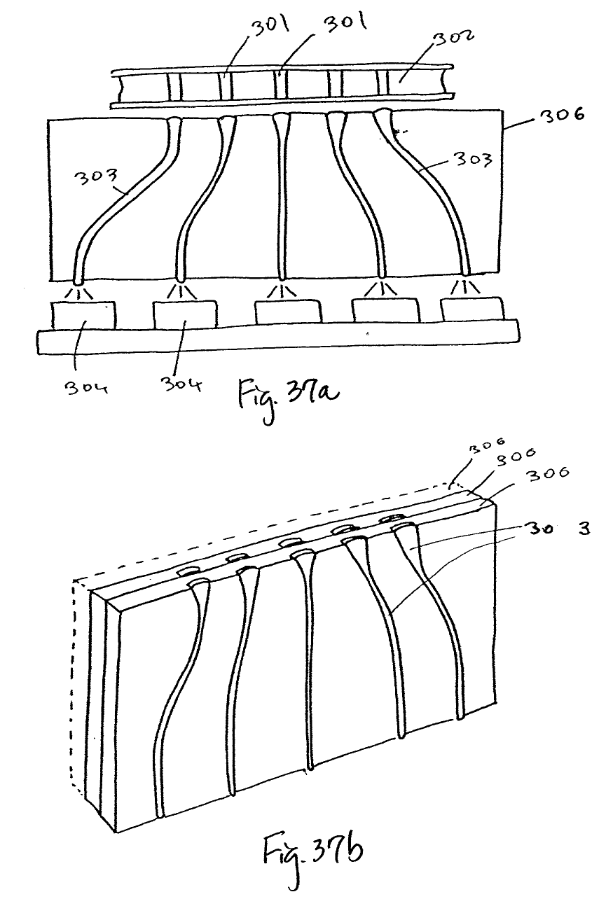

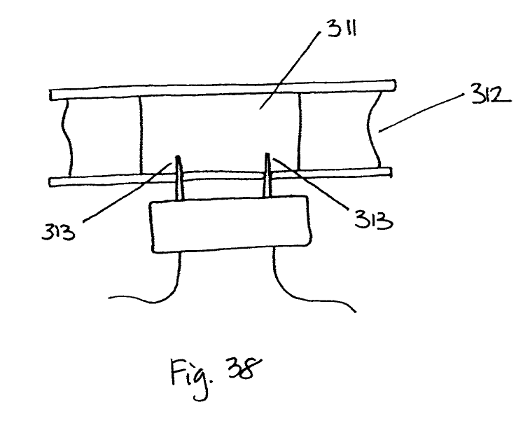

FIGS. 43a-d depict a novel capillary transfer device.

FIG. 43a provides a cross-sectional view of a storage, metering and dispensing device, shown in its nominal storage position. Fluid is stored within reservoir 111 with optional cap seal 112 at the top and plug seal 113 at the bottom. The reservoir and seals are preferably molded in polypropylene or other polymeric material resistant to solvents and biological reagents, or may be fabricated from stainless steel or other appropriate material.

In FIG. 43b, plug seal 113 has been removed in preparation for metering. This opens channel 114 into which fluid is drawn by capillary force or pressure. A droplet of fluid 115 forms at the opening. The droplet volume is controlled by the geometry, surface and material characteristics of the opening, together with the surface tension of the fluid, gravity and applied pressure if any.

In FIG. 43c, transfer plate 116 is positioned in contact with droplet 115. In this embodiment, the transfer plate contains a hole 117 that draws fluid from the drop to fill the hole. The transfer plate 116 is then removed, ready for dispensing, containing a metered volume of fluid defined by the volume of hole 107 together with menisci at either side of plate 116.

In FIG. 43d, pipette or other nozzle 118 is positioned above the transfer plate hole containing the metered fluid. This pipette is connected to additional fluid such as a reagent solution, buffer or water, or alternatively to gas or air. Additional fluid is injected through hole 117 in transfer plate 116, overcoming surface tension and expelling the metered fluid onto 119, such as may be a surface or receiving receptacle. The quantity of additional fluid may usefully be metered if it will chemically participate in some reaction or analysis to follow. Alternately, transfer plate 116 may be directly juxtaposed against surface 119 such that the metered fluid volume may be transferred directly, such as by capillary action optionally supplemented by gravity, or by wicking into the bulk of, or porous material on the surface of, 119.

### EXAMPLE 46

### Capillary Transfer Device for Storage, Metering and Dispensing

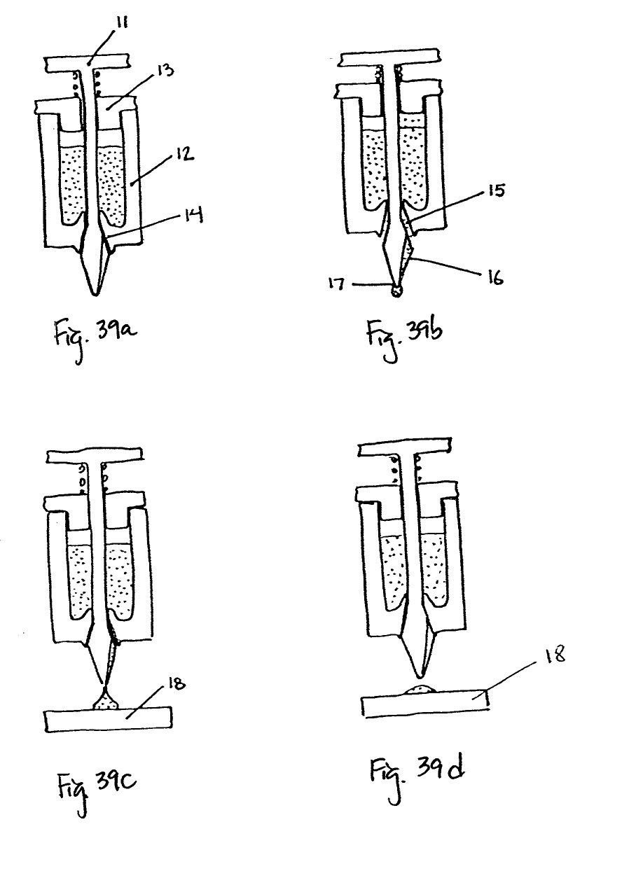

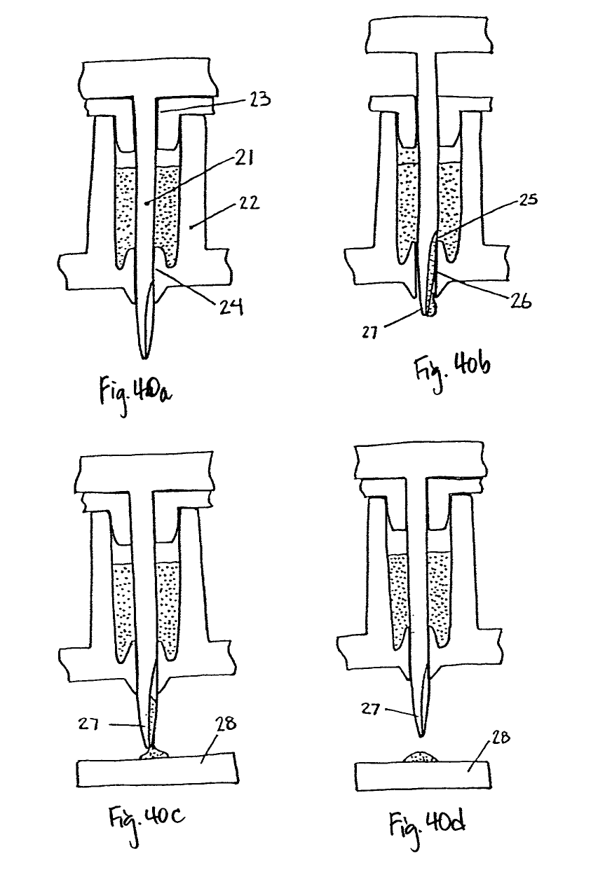

FIGS. 44a-c depicts an alternate embodiment of the above example.

FIG. 44a is a cross-sectional view of a storage, metering and dispensing device, shown in its nominal storage position. Fluid is stored in reservoir 121 with cap seal 122 at the top and sliding seal 123 at the bottom. The reservoir and seals are preferably molded in polypropylene or other polymeric material resistant to solvents and biological reagents, or may be fabricated from stainless steel or other appropriate material.

In FIG. 44b, sliding seal 123 has been moved in preparation for metering. This aligns channel 124 into which fluid is drawn by capillary force or pressure. A droplet 125 of fluid forms at the opening. The droplet volume is controlled by the geometry, surface and material characteristics of the opening, together with the surface tension of the fluid, gravity and applied pressure if any.

In FIG. 44c, the sliding seal 123 is returned to its nominal position, aligning the metered fluid with dispensing hole 126. Pipette or other nozzle 127 is positioned above the transfer plate hole containing the metered fluid. This pipette or nozzle may contain or be connected to additional fluid such as reagent, buffer or to gas or air. The additional fluid will be dispensed through the hole 124 in the sliding seal 123, which overcomes surface tension effectively expels and/or rinses the metered fluid onto 128, which may be a surface or receptacle.

### EXAMPLE 47

### Capillary Transfer Device for Storage, Metering and Dispensing

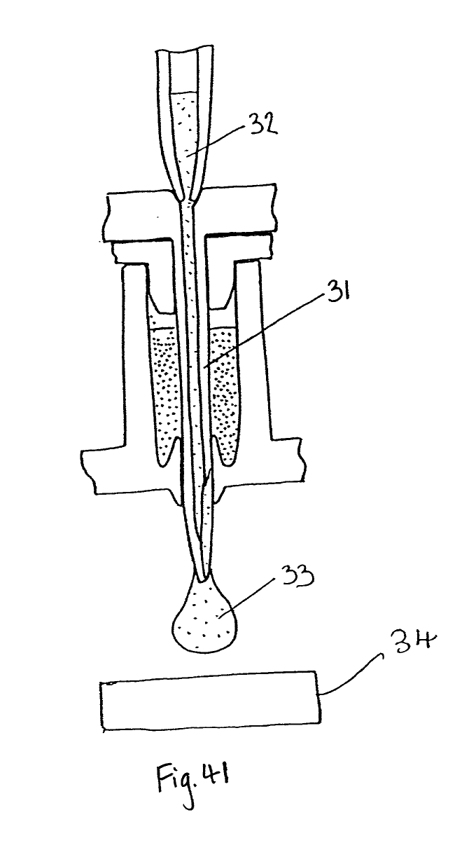

FIGS. 45a-c depicts an alternative embodiment of the above example.

In FIG. 45a, a storage device is fabricated from three sheets or layers, where middle layer 132 has one or more chambers fabricated between holes formed in the upper and lower surfaces. Upper sheet 131 and lower sheet 133 have one or more holes fabricated, that may, or may not, line up with holes on middle layer 132, depending on how the three layers are mutually positioned. Quantities of material, such as fluids, solids, powders or any combination, may be positioned within the storage chambers in middle layer 132 through holes in sheet 131 or 133 when either sheet is are positioned such that the holes are mutually registered with those in middle layer 132.

Once positioned in a chamber, material may be sealed and stored therein by movement of sheets 131 and/or 133 to move the holes out of registration. Material may be stored in multiple chambers, without disturbing material already sealed and stored, by appropriate motion of sheets 131 and/or 133. Alternately, chambers may be individually accessed by middle layer 132 being moved relative to sheets 131 and/or 133. Material may be removed from a chamber by mutual positioning of the three layers such that at least one hole on the upper or lower sheet registers with a chamber hole on the middle layer, such that material may exit such as by gravity. Alternatively, if both upper and lower sheets provide through-hole access to a chamber, removal of the material content may be assisted or controlled by applying a liquid or gas to positively expel the material.

The individual chambers may advantageously be of different shapes and volumes, such that, for example, known quantities of material may be stored by partially or completely filling chambers of known volume. Additionally, appropriate hole registration may permit two or more chambers to be accessed at once, such that multiple chambers may then be filled or emptied in parallel.

FIG. 45b depicts both rotary devices and two dimensional array devices, both constructed with three sheets or layers as above, where, respectively, rotary motion or linear motion in two axes are used to mutually position the three layers to achieve hole registration, or non-registration, as required to access, fill, or empty, individual chambers as above.

FIG. 45c depicts other chamber shapes whereby material, particularly liquids, may be stored in shaped chambers and may optionally use gravity to trap material in chambers without requiring a lower sheet to seal a chamber.

Other Devices

### EXAMPLE 48

### Storage, Distribution and Dispensing Using Laminar Sheets

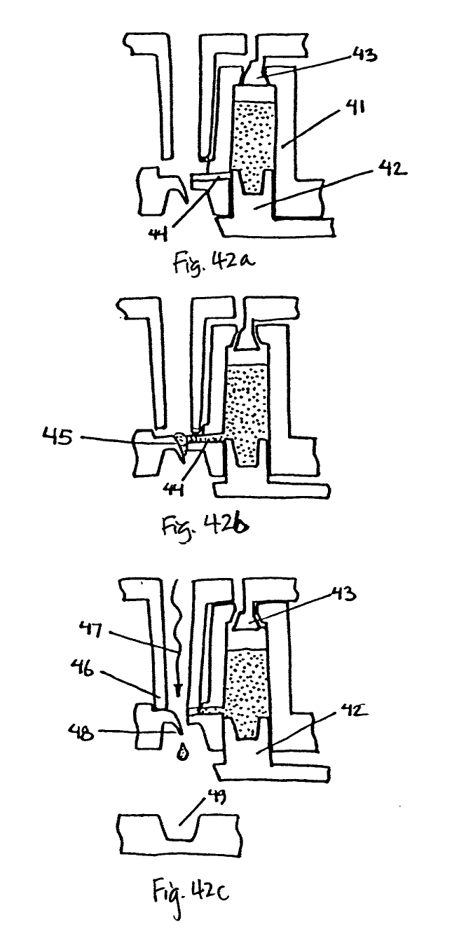

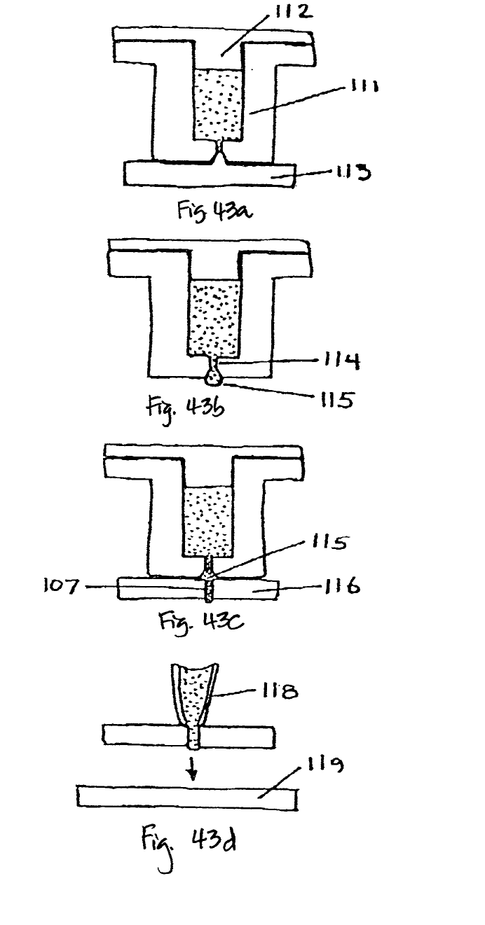

Existing pin transfer techniques are shown in FIG. 46a, where fluids are transferred from storage wells to e.g. a sheet substrate 51, such as solid polypropylene, treated paper or a spunbonded material such as Dupont's Tyvek (spunbonded polyethylene). Such materials may be resistant to DMSO and other solvents commonly used in assays and are readily available in sterile, medical grades. In some cases the fluid will be allowed to evaporate, as shown in FIG. 46b, leaving solute residue 52 in its place.

The present invention provides that multiple copies of each array of solute or other material may be made, and then the sheets containing cells of sealed said material optionally may be stacked in a storage container (not shown). The storage container may optionally resemble an existing deep well plate, to be easily handled by existing robotic techniques. Such cells may be indefinitely stored with their material content and may subsequently be pierced, cut or ruptured to release their material content.

In FIG. 46c, second sheet 53 is placed over the top of sheet 51 and sealed by adhesive, thermal or other means around each area of material using fixture 54. Separation of cells and mutual isolation of their contents may usefully prevent cross contamination between cells and also may advantageously protect such stored materials from exposure to fumes, oxygen, moisture and light, etc.

In FIG. 46d, sheets 51 and 53 are punched with die or similar means 55 which deposits opened cells to effectively dispense stored material such as into wells 56 or other locations or devices. The die diameter may optionally and advantageously be smaller than the sealed cell diameter, such that the cells are thereby unsealed and separated and the stored materials therein are released. Optionally, material stored within the cell may be dispensed into a liquid 57, and/or liquids may be added after such dispensing, such that the material may participate in a chemical reaction such as an assay, or optionally be detected before, during or after a chemical reaction. Although element 55 is depicted as a punching die, alternate cell piercing means may be used such as a probe, knife or other object able to cut or rupture cells such that their contents are released.

Multiple such storage cells may be created e.g. in an ensemble or array. Advantageously, the means for sealing and subsequently unsealing such cells may operate on multiple cells simultaneously or sequentially. Ensembles of such cells may be manipulated such as by physically dividing the ensemble of sealed cells (films 51 and 53 with multiple materials 52 sandwiched therein) into smaller ensembles, or even individual cells, that may then be independently stored, sorted and processed such as by unsealing.

Such cells may be used to store and release material, or may be used as containers for chemical reactions such as from heating the cell content material, or for weighing the cell and contents and inferring the stored material weight or for detecting the cell contents by any means able to measure the properties of the cell contents. In addition, the cells may be really or virtually labeled, such as individually, or in ensemble, or indirectly such as from their position in an ensemble, such that each cell may be uniquely identified and the location, storage, management and subsequent processing of cells is facilitated thereby. Advantageously, robotic means may be used to efficiently and rapidly manipulate such cells individually or in ensembles of any size or organization.

### EXAMPLE 49

### Storage, Distribution and Dispensing Using Preformed Sheets

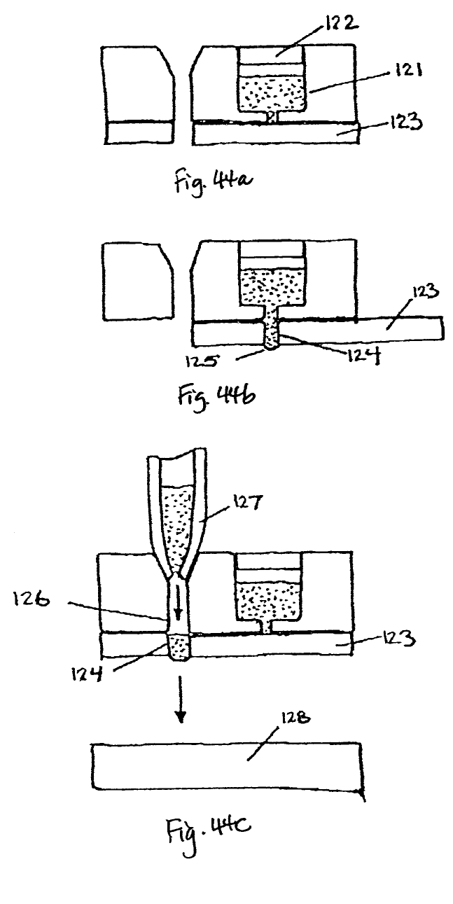

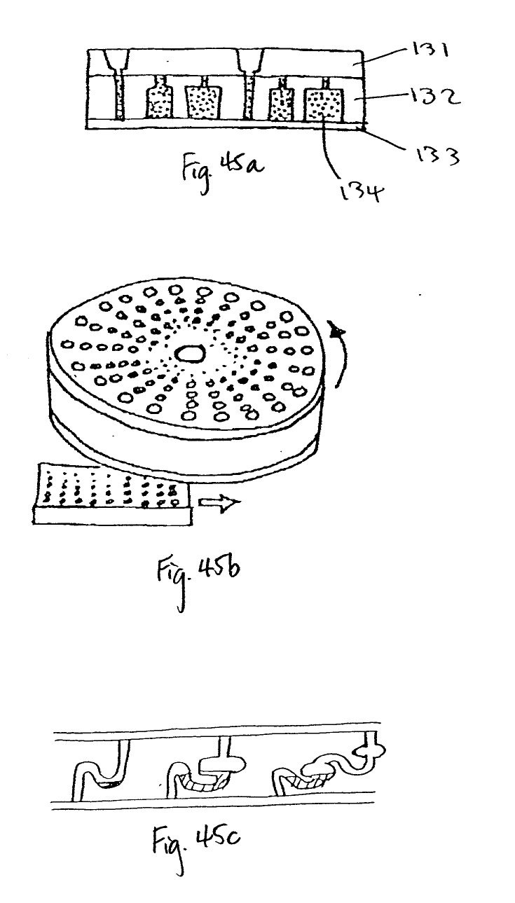

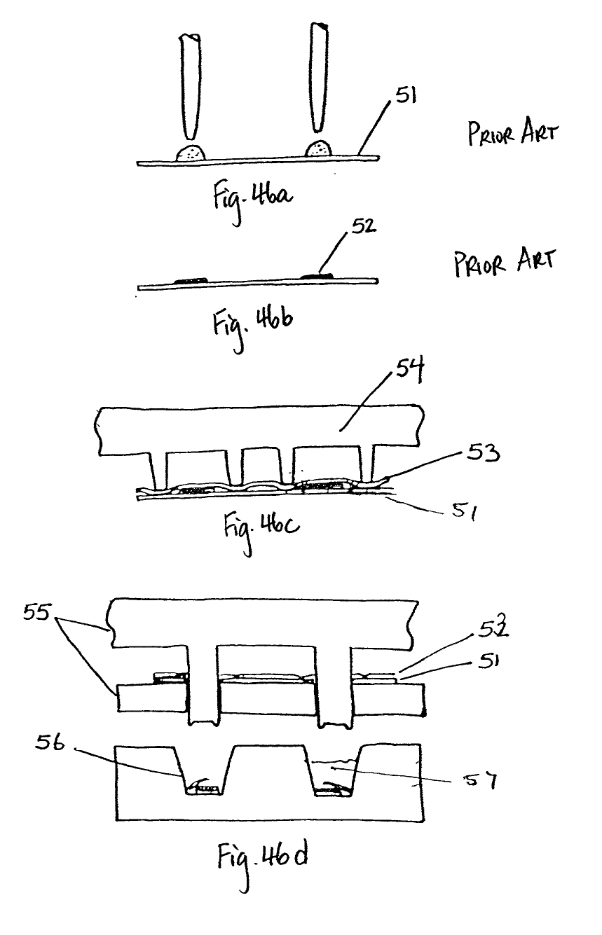

In another embodiment depicted in FIGS. 47a-c, a layer, such as formed from polypropylene solid sheet or woven material, is heat formed or otherwise shaped to create shaped volumes 61 such as miniature assay wells. Reagents or other materials are added in liquid, solid or powder form or any combination, then the top of said shaped volume is sealed with Tyvek or another appropriate sheet material 62, such as thermally, with adhesive or by other known means. The seal may optionally be achieved after a delay for a process to occur such as a chemical reaction or evaporation of one or more liquids, leaving a dried residue. Such sealed volumes or cells may be so implemented individually or in small numbers, or in an ensemble or array. Material contained within a cell may be modified, such as by thermally or photolytically induced chemical reaction, and/or may be detected by a variety of known optical and other means.

To release the stored material, sealed sheet 62 may be peeled away entirely or in part, such that the material in the shaped volume is exposed and may be accessed. Such peeling away may be to unseal or uncover just a single storage volume, or several such volumes, or all or part of an ensemble or array of such volumes. Thereafter, a chemical reaction such as an assay may be performed directly in each well. Alternately, the released material could be moved elsewhere for the purposes or chemical reaction, chemical assay, and/or detection, optionally being dissolved or diluted with liquid beforehand.

Optionally, bottom sheet 61 may be retained, and held flat such as with vacuum platform 63, to improve holding, registration and manipulation accuracy. Such volumes, particularly in ensembles, may advantageously form the basis for a low cost assay platform, replacing traditional assay plates.

FIGS. 47d-e illustrate the basis for an invented new assay methodology. Using techniques as above, reagents are packaged in sealed thermoformed wells. Various chemical compounds 65, or other reagents, could be packaged separately as shown or integrally in layers. To perform an assay or chemical reaction, the bottom sheet is placed in a tray 66 with the wells 64 and 65 aligned, and a die, probe or other means 67 is used to punch through or rupture the upper layers. This advantageously prevents cross contamination between the storage cells 65 and adjacent wells 64. Optionally, the top layer 69 has superior elasticity properties and therefore simply stretches around the die 67, whereas the lower layers of less elasticity or strength rupture and dispense the contents of sealed cells 65 into wells 64. This advantageously will prevent contamination of the die 67, which may thereafter be used for subsequent operations without cleaning. After an assay or other chemical reaction has been conducted, optionally the results may detected by detector 68 such as an optical device or by other means.

### EXAMPLE 50

### Storage and Dispensing Using Spacers and Sheets

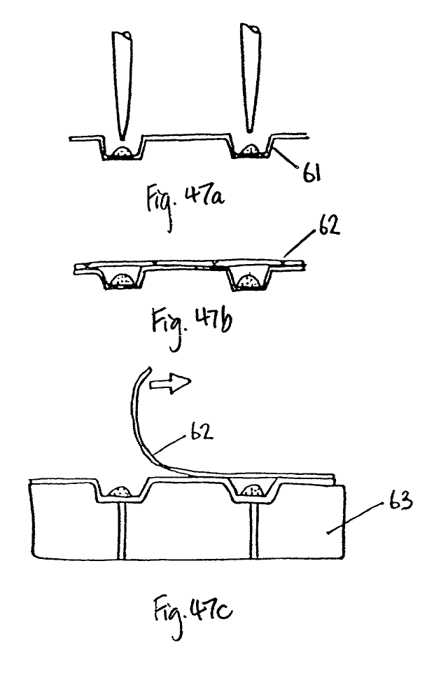

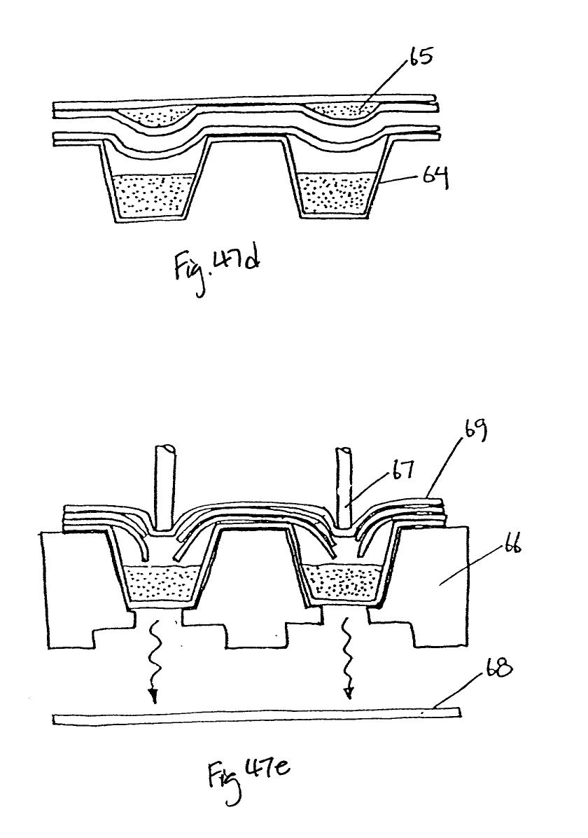

Further forms of this version of the invention may be readily constructed. FIG. 48a depicts the case where a spacer 74 is positioned between the two sheets 72 and 73, and one or more holes 71 formed through the spacer conveniently define the volume of a storage cell between the two sheets, where multiple such holes may conveniently be arranged in an ensemble or array. Each such storage cell may contain a gas, liquid, solid or powder or any combination thereof.

FIG. 48b depicts an alternative design with three sheets and two spacers forming a two-tiered device with cells at two levels, optionally arranged to register vertically as shown. Further, additional tiers may be added similarly.

A probe or other actuator 75 may be used to push through each cell and to expel the contents. The probe will then puncture two or more registered cells in sequence such that the contents are all expelled, such as onto surface 77, or e.g. into one or more wells, after which said contents may usefully be mixed and then optionally interact or take part in a chemical reaction, optionally with additional reagents prepositioned on 77 or added subsequently, and optionally be detected.

Devices Using Porous Materials

### EXAMPLE 51

### Slice and Dice Cassette for Compound Metering and Dispensing

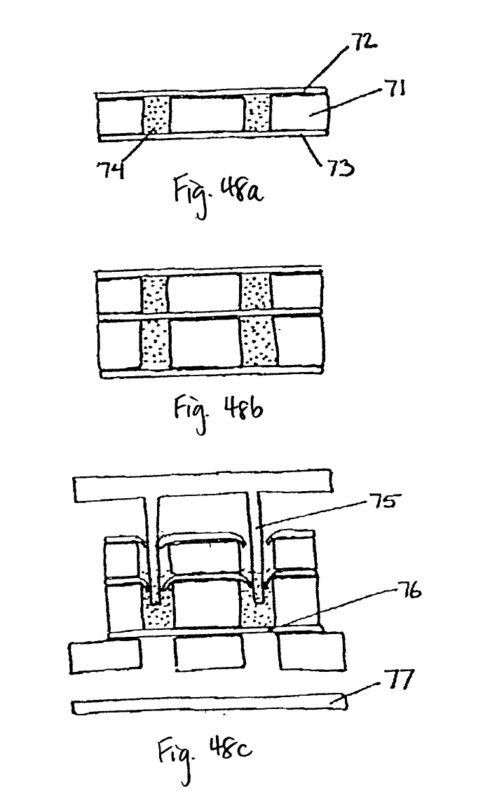

In FIG. 49a, microcapillary tube or rod 81 of porous material such as “Porex” (for example, porous polypropylene beads heat-fused together) is placed in reservoir 82 containing a fluid containing one or more compounds in solution. The fluid is drawn into the tube or rod by surface tension and capillary action.

In FIG. 49b, rod 81 is placed in chamber 83, optionally sealed to protect the fluid and dissolved solutes from oxygen, moisture and light. The fluid and dissolved components may advantageously be uniformly disposed along rod 81, so that to meter and dispense a known amount of same, the rod can be cut to an appropriate length. At the bottom of chamber 83 is sliding sheet 84 with hole 85 corresponding to chamber 87. Normally, hole 85 is not aligned with chamber 83. During metering, hole 85 is aligned with chamber 83 as shown, such that rod 81 may be advanced, optionally by a known distance 86.

In FIG. 49c, the sliding sheet 84 is moved again such as back to its normal position, slicing off the protruding rod section 86 to dispense same. The released slice may fall optionally into e.g. a well or other receptacle or surface 87 below.

Advantageously, several such rods may be placed in an ensemble or array and optionally fabricated or stored together such as in a cassette or other device. Each rod resides in a separate sealed chamber, and each sliced section may be released or dispensed to a separate location, such as a separate well or other container, advantageously preventing cross contamination. Alternately, by individual control the motion of chamber 83, or of well 87, and particularly when one or both are organized as ensembles, the deposition of slices to any destination location may be individually controlled. Further, two or more slices 86, optionally from differing chambers 83 may be deposited at any location 87. In addition, multiple slices from multiple chambers may be deposited at multiple locations, such that some or all chambers 83 may service some or all locations 87 and combinations of slices may generally accumulate at any such location.

The above device may be used for the preparation, storage and metered dispensing of prepackaged materials such as chemical compounds and reagents. In addition, one or more slices may be deposited onto a waiting sheet, optionally in an ensemble or array, then sealed for storage, sorting, management and later use such as by means described above.

### EXAMPLE 52

### Porous Beads for Compound Metering and Dispensing

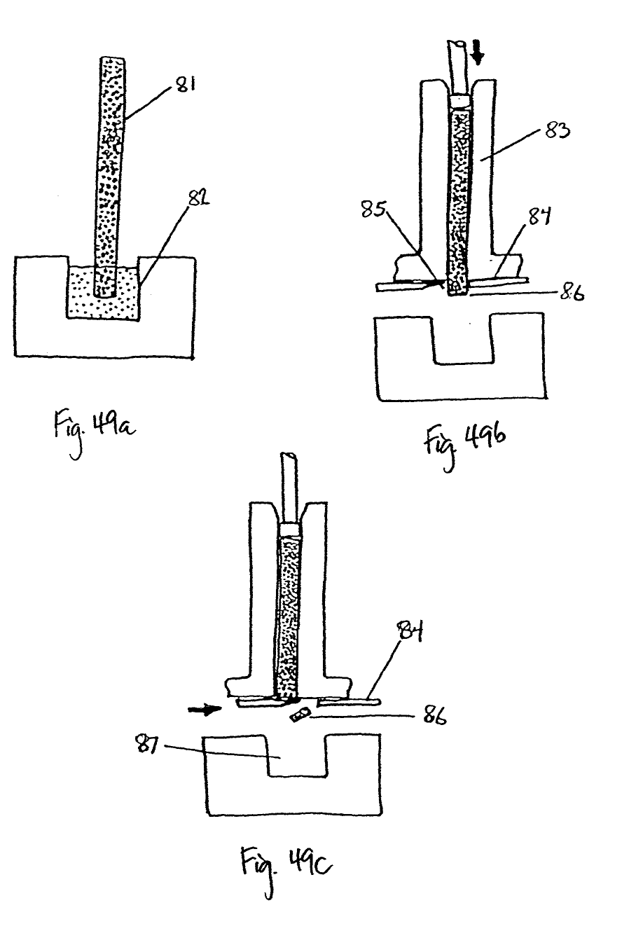

In FIG. 50a, a known quantity of porous material such as sintered beads 91 is placed in reservoir 92. Solution 93 containing chemical reagents is added to the reservoir and is absorbed by the porous beads, with the quantity, size and porosity of the beads determining the volume of fluid that may be so absorbed. The volume of fluid absorbed per bead, and concentration of chemical reagents in solution, may then determine the quantity of reagents contained in each bead.

In FIG. 50b, the beads 91, with known chemical reagent content, are transferred to preformed wells 94 in sheet material 95. The beads are then covered with a second sheet 96, and are sealed thermally with adhesive or other known means, providing for their storage at specific locations and optionally protecting the reagents exposure to e.g. fumes, oxygen, moisture and light. Beads may be stored in each storage cell either individually or as a plurality. Such storage cells may be fabricated individually, or multiple such cells may be fabricated from sheets 95 and 96, such as in one or more ensembles or arrays. In addition, by the use of three or more sheets (not shown) two or more storage cells may be similarly created with vertical registration between cells.

As shown in FIG. 50c, dispensing the beads is affected such as with blunt probe 97, which ruptures at least lower sheet 95 and forces the cell contents to fall into receptacle 98. To prevent contamination of the probe 97, top sheet 96 may advantageously be prepared from material having better stretching or strength characteristics than the material of bottom sheet 95, such that top sheet 96 will merely stretch with the advancing probe rather than rupturing.

In an alternative embodiment, porous beads are placed dry in one or more, or optionally an ensemble or array, of such shallow wells pre-formed in a sheet. A multi-channel pipette device (not shown) transfers reagent solutions to the beads within the wells. The wells are then closed with a second sheet sealing each well individually, such that reagent samples may be individually stored then later dispensed as described above.

### EXAMPLE 53

### Porous Beads for Compound Metering, Storage and Dispensing

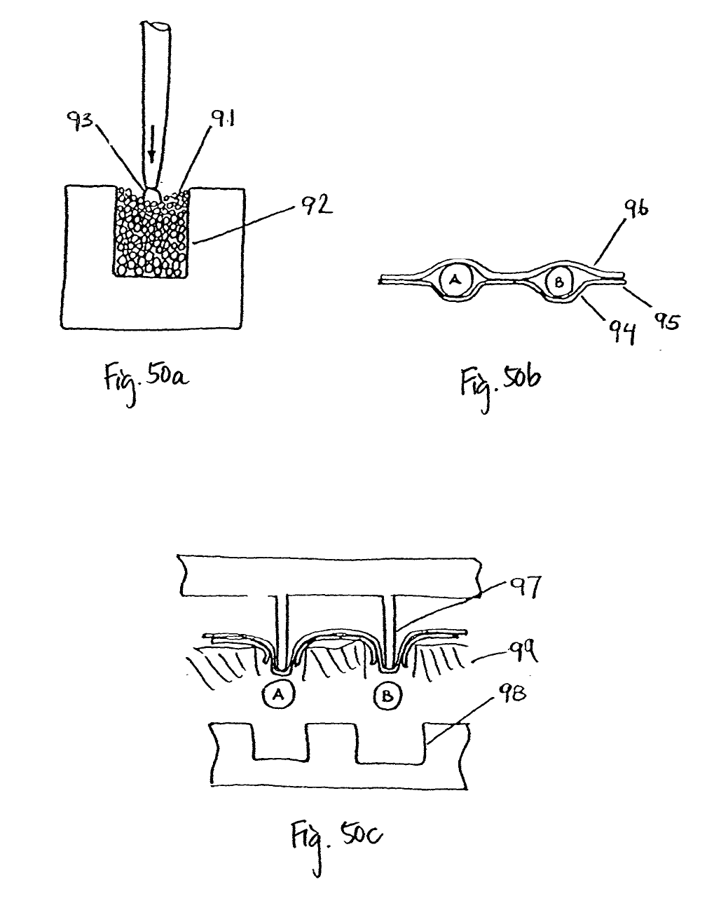

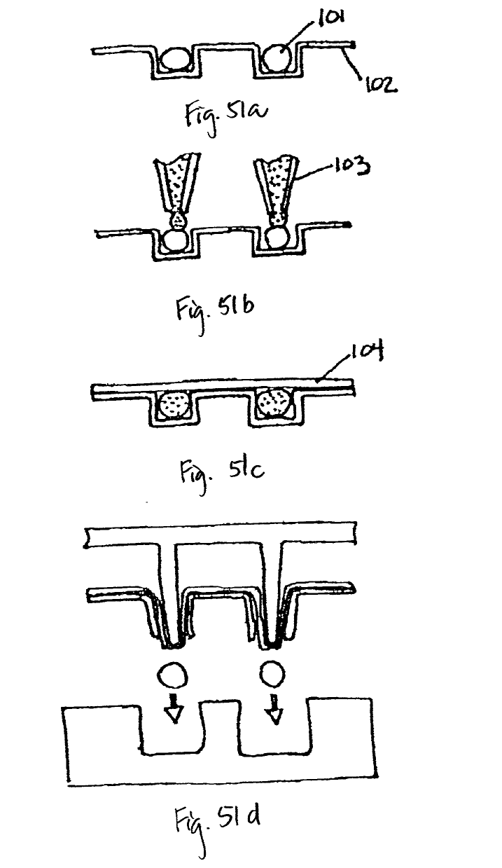

An alternative embodiment of the above is depicted in FIGS. 51a-d. Per FIG. 51a, porous beads 101 are placed in wells formed in sheet 102. One or more such wells, each containing one or more beads, may be fabricated individually or as an ensemble or array.

Per FIG. 51b, pipette device 103 transfers reagent solutions to the beads within the wells. One or more quantities of fluid, optionally including chemical reagents, buffer solutions or preservatives may be applied to each or several beads, with different beads optionally receiving differing combinations of fluids from one or more pipette devices. With the wells arranged in arrays, such dispensing may advantageously be conducted with multi-pipette devices, particularly when robotically controlled. The deposited contents of the wells may then optionally be dried, lyophilized or chemically reacted.

As depicted by FIG. 51c, the wells are then sealed with second sheet 104, forming one or more storage cells containing beads with chemical reagents, etc.

Per FIG. 51d, the cells are ruptured and their bead contents are released and dispensed as described above.

### EXAMPLE 54

### Porous Storage and Transfer Sheet

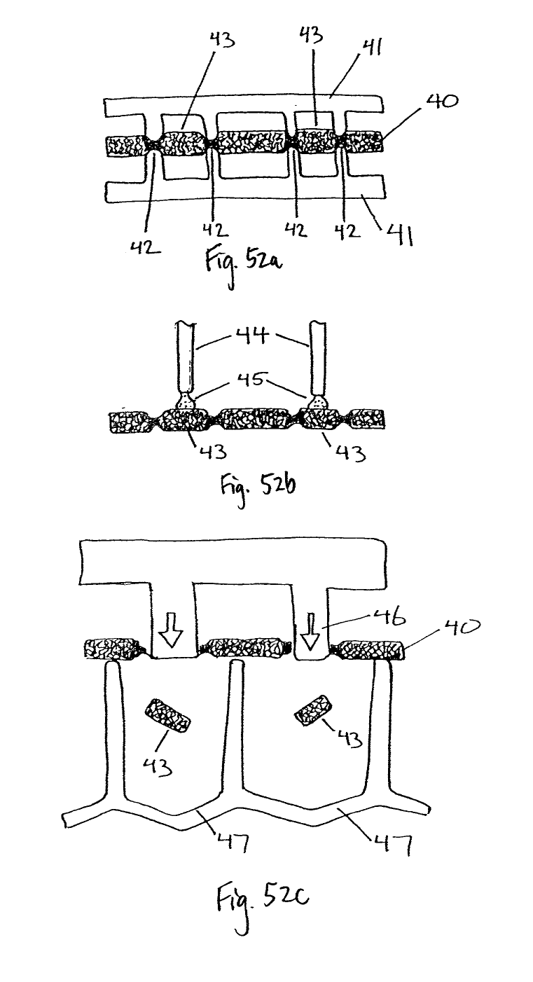

Per FIG. 52a, a porous sheet material 40, such as woven, spun-bonded or meltblown polypropylene is processed under heat and pressure from a die 41 to form non-porous regions 42 surrounding porous regions or cells 43. The selectively applied heat and pressure fuse the pores, creating a matrix of solid, non-porous material. The interstitial porous cells 43 are thereby isolated from one another, preventing sample migration or intracellular cross-contamination.

Per FIG. 52b, liquid samples 45 are transferred to the porous cells 43 with a multi-channel pipette or pin transfer device 44. The liquid samples 45 are drawn into each cell 43 by capillary action. The amount of fluid transferred is controlled by the amount of fluid present on the pin or pipette tip, by timing, gravity and pressure and by the porosity and surface energy properties if the porous sheet material 40 or any combination. Alternately, the total stored volume may be controlled by the porosity and volume of the porous cell 43.

Certain assays may require a higher concentration of sample be stored in the cell, such as to ensure that adequate signal strength is generated for detection. In this case, the fluid solvent carrying the desired compound in each cell can be allowed to evaporate, leaving dried down compound in the interstitial spaces of the cells. Once the solvent has evaporated, additional liquid sample may be added and allowed to evaporate again. This process may be repeated several times to increase the quantity of solute within the available cell volume.

Alternately, several different samples types may be stored in the same cell, by allowing the solvent to evaporate between applications. This pooling and multiplexing may be used such as for high thruput screening of drug candidates to reduce the number of tests required to initially identify promising candidates. If a particular cell registers a positive result, the test may later be repeated with its individual samples in separate cells to identify the responding compound.

Sample transfer to an assay plate can occur as described in Example 2, or the assays may be performed on the surface of the cell 43 in small drops of reagent and/or buffer (not shown). Alternately, as shown in FIG. 52c, the porous cells 43 can be cut with a die 46 directly into corresponding assay wells 47. Advantageously, an additional, layer optionally disposable after single use, may be positioned between die 46 and sheet 40 (not shown) such that the die is not contaminated by contact with the sheet material and/or its contents. Die 46 can advantageously use a mating half between the sheet 40 and the assay plate 47, or plate 47 may be so fabricated, to improve the quality and accuracy of the die cutting, thus reducing the frequency of partially cut cells 43 or “hanging chad”.

Fabrication and Manufacturing Considerations:

Materials:

A common application for this device will be the storage of large numbers of individual molecular compounds, typically dissolved in 100% DMSO (Di-Methyl Sulfoxide). The material most commonly used for storage of these materials is polypropylene, for its chemical resistance to DMSO. However, other materials may be used and are all covered by this invention, and include other plastics, silica and all glass materials, silicon and other semi-conducting materials, metals (optionally passivated), ceramic materials, inorganic material, non-synthetic organic material and naturally occuring material.

Seals are likely to employ multiple layers of film and/or foil to achieve the following properties: chemical resistance, heat staking or sealing, low oxygen permeability, low moisture permeability, low UV transmission. A likely candidate is polypropylene and aluminum multi-layer foil with the polypropylene in contact with the storage and transfer sheet. However, other materials or combinations as may be known to those skilled in the art are included in the present invention.

The material for the reaction cells will be chosen based on the type of assay that will be performed, and the detection techniques used. Common materials will include white, black or clear polystyrene, natural or clarified polypropylene, and polycarbonate

Hole Geometry:

Hole geometry is an important element to ensuring accurate sample metering during loading. Entry and exit chamfers or steps will cause fluids to preferentially locate in the center of the sheet, away from sealing surfaces and potential wicking. Additionally, the surface of the sheet and/or sealing material may be treated to make them more hydrophobic than the hole inside surfaces, which may be accomplished by any known surface treatment means. Alternately, hole inside surfaces may be made hydrophilic, such as by oxygen plasma surface treatment or with microscopic surface features such as grooves.

Process Control:

Sheet thickness variability affects the volume of the microdrilled holes, causes variability between sheets and between holes or hole clusters. One solution is to monitor sheet thickness in real time during fabrication, and to modify the hole diameter as needed to maintain a constant hole volume. For example, as sheet thickness decreases from the nominal value, the hole diameter increases slightly to compensate. Likewise, as sheet thickness increases, the hole diameter decreases.

Cell Fabrication:

Cells and cell arrays may be fabricated, depending on the material or combination of materials utilized by, such techniques as injection molding, compression molding, thermoforming and embossing, machining and micromachining, stamping and forming, die cutting and slitting, perforation, lamination and layering, weaving and fiber bonding, brazing and welding (thermal, ultrasonic, induction), adhesives and mechanical fastening.

Cell Shaping and Topology:

Cells details and/or surface characteristics may by created or enhanced to optimize performance by, e.g., etching, such as by chemical and/or laser treatment; by photolithography; by polishing, lapping and grinding; by preparation of coatings; by plating and vapor deposition; by plasma surface modification; by embossing and by printing, including screen, pad, transfer and ink-jet techniques.

Use:

Samples may be transferred to a device according to the invention by pipette, pin transfer, piezo-type dispenser, syringe pump, direct synthesis in cells, filtration through device, mechanical or robotic transfer of substrate such as polystyrene beads, or immersion (for bulk transfer of single sample to many cells).

Cells and cell arrays may be sealed for storage by using adhesive sheet or foil, heat welding, ultrasonic welding, press-fit plugs or gaskets, vacuum packing in bag or other enclosure, layering of other cells or cell arrays, self-skinning material application, or no seal-liquid samples allowed to dry down,

Chemical reagents may be contained within a single cell, or contents of adjacent or co-axial cells may be mixed, or cell contents may be dispensed into external reaction vessels by seal removal by peeling, tearing, suction, etc. Also slitting or cutting of cells to expose and/or remove contents may be used as well as mechanical rupture of seals with a pin, probe or other device, mechanical rupture by impulse or pressure (fluid or gas), distortion of flexible outer layers, rupturing inside barriers, stretching, twisting or compression, rupturing inside barriers, die cutting portion of cell containing sample into external vessel, puncture and dispense reagent, buffer, etc. into or through each cell, applying heat or other reaction-inducing energy, melting frozen components, adding water, buffer or other solvent, diluent or reagent solution, and bringing two or more devices together, enabling fluid wicking and mixing.

A variety of chemical reactions and assays together with all appropriate detection methods are supported by the invention and may occur in any of the cell types described or in variants or combinations such as, homogenous chemical reactions, heterogenous chemical reactions, chemical reactions using beads or other solid substrates, multi-stage chemical reactions, combinatorial chemistry multi-stage chemical reactions, immunoassay reactions, agglutination assays, sandwich type and assays, enzyme amplified and ELISA types immunoassays, blood typing assays, protein binding assays, nucleic acid hybridization assays, aptamer binding assays, PCR based amplification assays, LCR based amplification assays, high throughput screening assays, drug discovery assays, other chemical reactions with detectable products, detection of material not chemically reacted.

Temperature can be controlled by immersion in a temperature controlled fluid or gas, radiation heating and/or cooling, thermal coupling to a temperature controlled body or reservoir, maintaining at one or more defined temperatures for defined times, heating or cooling at defined temperature rates.

Exemplary detection means include methods such as monitoring of sample radioactivity; thermal detection such as from an exothermic or endothermic reaction; optical absorption in the IR, NIR, visible or UV regions; optical emission techniques such as chemiluminescence or phosphorescence; optical fluorescence including amplification by enzyme action; and potentiometric and/or amperometric electrochemical detection.

Alternative Embodiments:

In summary, the invention includes making or manufacturing storage cells as herein described, inserting a volume of a first sample into said cell, optionally adding an inert substance to the cell's remaining volume, sealing a cell either empty or with a sample volume therein, treating the surface of the cell, its substrate or its sealing means, identifying such cell individually and/or as part of a cell ensemble, storing such cell individually or as part of an ensemble, managing the storage and retrieval of such cell from its identification, retrieving a cell, or ensemble of cells, containing a sample volume, optionally adding one or more additional samples to the storage cell, inducing one or more chemical reactions to occur within said cell, optionally using applied heat to assist with such chemical reaction, detecting whether a chemical reaction has occurred within said cell, detecting whether a desired or intended chemical reaction occurred, detecting the level of reaction which occurred or the quantity of product, making such detection by a range of optical means, making such detection by a range of electrical means, resealing the cell after addition of a second or subsequent sample, and safely disposing of cell, individually as an ensemble, sealed or unsealed.

Devices and methods as described above represent merely preferred embodiments of much broader concepts, and alternative embodiments are possible such as the utilization of alternative materials, dimensions and other potential uses and applications of stored and dispensed solids, liquids and mixtures, where all such additional embodiments as may be perceived by those skilled in the art are all included herein by reference and explicitly made part of the current invention. In particular modifications such as the use of 0-rings instead of direct contact seals are specifically included. Furthermore, although each example contains a number of discrete elements, other combinations of all the elements herein described are included by reference as other combinatorial examples supplementing the preferred embodiments as here explicitly described.

Although the examples imply that a single phase of delivering material to a storage cell may occur, the invention also includes more than one stage of such addition. This may include the use of more than one pipette tip, pin transfer device or other known means. It may also include one or more separate additions of one or more physical forms of material, including solids, fluids, powders, gels, emulsions, slurries, or beads. This may include one or more drying, lyophilization or other stabilization stages, together with one or more cell sealing and/or unsealing stages and one or more periods of storage optionally at reduced temperature.

Furthermore, there may occur more than one stage of chemical reaction. This may be achieved such as by merely heating or irradiating material within a cell, or by adding additional material such as fluid and inducing or allowing a chemical reaction to occur more than once. Optionally, material may be decanted from a cell (such as removed by a pipette tip, pin transfer device or other known means) between stages, with additional material optionally being added before a subsequent stage.

The invention covers homogenous and heterogenous chemical reactions. In particular, the use of beads or other solid particles or the surface of the cells themselves is explicitly included herein, optionally with chemical entities attached to one or more surfaces. Heterogenous reactions are facilitated by means including dispensing beads or other solid particles into a cell such as by a pipette or other known means. Some or most of the fluid may be removed from a cell such as by a pipette or other known means, such that fluid is removed while the beads are caused to remain in the cell, such as by causing the beads or other particles to collect in the bottom or other region of the cell assisted by e.g. gravitational, magnetic or other means, following which additional fluid may be dispensed. Such additional fluid may be applied to wash the beads or other particles or cell walls, which may be facilitated by agitating the fluid including by cycling it between the cell and the volume of the pipette tip as described above, following which the washing fluid may be removed by permitting the beads or particles to settle as above, following which some or most of said fluid may be removed.

Chemical reactions may occur after the addition of additional fluids to a cell already containing fluid, and/or after the addition of fluids to coated beads other particles or coated cell walls. Chemical reactions may also occur after the addition of material in non-fluid physical forms such as solids, powders, gels, slurries, etc. to such cells. In all cases, chemical reactions may be induced or assisted by all known means applicable to such chemical reactions, including without limitation by the passing of time, by applied heat optionally on a heat/cool cycle such as required for the polymerase chain reaction (PCR) or similar, by applied light or other electromagnetic radiation, and/or by the inclusion of catalysts (including enzymes) within the reaction mixture or part thereof, both to catalyze one or more chemical reactions and/or to amplify one or more chemical substance such as a fluorophore for detection.

Other physical and/or chemical extensions to the examples, as may be perceived by those skilled in the art, are all covered by the present invention.

The examples above describe cells in sheets or other laminar materials, fabricated in any shape or pattern with any sets of dimensions. For some embodiments of the invention, it is advantageous to fabricate such sheets in the formats as used with 96, 384 and 1536 well plates, permitting cells and sheets per some embodiments of the invention to be used with apparatus, equipment and instrumentation already compatible with such commercial formats. Thus, embodiments of sheets per this invention may be fitted to or utilized by such apparatus, either directly or by relatively minor modification to such apparatus, such as with a modified mounting, or by fitting within a carrier then fitting into unmodified apparatus. Thereby, sheets containing cells such per embodiments of the present invention may be used within laboratories, and utilizing apparatus, etc., similarly to existing 96, 384 and 1536 well plates.

Embodiments of the present invention may be used as part of high throughput screening (HTS) procedures for assisting with drug discovery. Such sheets and cells may be used initially for holding and storing compounds such as obtained from a compound library. For example, each such cell may be charged with a sample of one compound such that an entire sheet may contain 96, 384, 1536 or some other number of such compounds. Such a sheet may be charged with such compounds from the compound library, then sealed and optionally stored before being used for chemical reactions such as at a different location. Any robotic or other means may be used per the present invention to charge, seal, store such sheets, either singly or in ensemble, and to unseal and facilitate chemical reactions, including without limitation single and multi-channel pipetting and pin transfer devices, heat sealers and robotic moving devices, etc. The storage sheets, pipette tips, or fluid transfer pins may be easily disposed of thereafter without contamination of apparatus, equipment, detection instruments or robotic apparatus.

For HTS embodiments of the invention, a sheet with cells may be charged with different library compounds that are to be reacted with target compounds. After optional storage as above, one or more such stored library compounds may then be reacted with the same target, such as with apparatus dispensing the same target material into multiple cells of one or more sheets, such that an indefinitely large number of compounds from a library may be automatically tested against a single target compounds, or vice versa.

The occurrence of chemical reactions, and/or identification of the products of such reactions, may be detected and optionally measured by all available means and apparatus, including particularly optical detection equipment such as compatible with 96, 384 and 1536 well devices that, by means above, will work with appropriate embodiments of the present invention.

Although many of the examples above are described in terms of simple closed cells, alternative embodiments exist where the cell is open in that one or more sections of the cell wall may be missing providing physical access to the interior and optionally the contents of the cell. Also, more complex cell designs and fabrication techniques may alternatively be used with any embodiment. Although not all are explicitly described, all such alternative embodiments are explicitly covered by the present invention.

The above examples describe the addition of fluids to stored materials. In addition to the simple case of a single fluid addition as covered in the examples, this invention also explicitly covers more than one fluid addition optionally using more than one pipette or other means, optionally with more than one mixing and solution step by turbulent fluid flow and optionally more than one mixing step by fluid retraction into the dispensing means and one or more washing steps.

The above embodiments give discrete combinations of details in each case. Other combinations of details are possible, including more or less complex embodiments and different combinations of discrete elements of the invention. Also, other features as known to those skilled in the art may be additively used. Such alternative embodiments are explicitly included in the present invention.

While the present invention has been described in conjunction with a preferred embodiment, one of ordinary skill, after reading the foregoing specification, will be able to effect various changes, substitutions of equivalents, and other alterations to the compositions and methods set forth herein. It is therefore intended that the protection granted by Letters Patent hereon be limited only by the definitions contained in the appended claims and equivalents thereof.

What is claimed is:

1. A microdevice comprising
a substrate comprising an upper surface, a lower surface and a channel in said substrate extending between said upper surface and said lower surface, said channel having an end in each of said upper and lower surfaces, said channel being of a sufficiently small diameter to maintain a fluid inside said channel by capillary action, wherein either a portion of said upper and lower surfaces around said each channel end is recessed or each end of said channel has a diameter sufficiently larger than that in the remaining portion of said channel away from said channel ends that any fluid substance in said channel is caused by capillary action to locate preferentially in said remaining portion of said channel.

2. The microdevice of claim 1, said device comprising a plurality of channels.

3. The microdevice of claim 1, wherein the length of said channel is the shortest distance between said upper surface and said lower surface of said substrate.

4. The microdevice of claim 1, wherein the length of said channel is longer than the shortest distance between said upper surface and said lower surface of said substrate.

5. The microdevice of claim 1, wherein said channel enlarges in said substrate to accommodate a reservoir portion of said channel.

6. The microdevice of claim 1, wherein the end of said channel in said upper surface is displaced from a point in said upper surface that is on a line orthogonal to said lower surface at the end of said channel in said lower surface.

7. A microfluidic system comprising the microdevice of claim 1, said system further comprising a sealing material for sealing one or more of said channel ends.

8. The system of claim 7, wherein said sealing material comprises one or more sealing sheets for covering a surface of said substrate in said device and sealing thereby said one or more channel ends.

9. The system of claim 8, wherein said system further comprises a sealed receptacle filled with components for an assay.

10. The system of claim 9, wherein said device comprises a plurality of channels and wherein said receptacle comprises multiple receiving elements.

11. The system of claim 10, wherein said device comprises a plurality of channels and said receptacle comprises multiple receiving elements and wherein, further, the ends of said plurality of channels in the lower substrate of said device are in a pattern corresponding to the pattern of receiving elements in said receptacle.

12. The system of claim 9, wherein said receptacle further comprises a feature to enhance optical detection.

13. The system of claim 12, wherein said feature to enhance optical detection comprises a lens for focusing emitted or transmitted energy.

14. The microdevice of claim 2, wherein the ends of said plurality of channels in said lower substrate are in a pattern corresponding to the pattern of receiving elements in a receptacle.

15. The microdevice of claim 14, wherein said receptacle is a microwell plate.

16. The microdevice of claim 2, wherein said channels are organized into clusters of channels.

17. The microdevice of claim 1, wherein said device comprises polypropylene.

18. The microdevice of claim 2, wherein said substrate is shaped to cause portions of said substrate comprising said channels to project away from portions of said substrate not comprising said channels.

19. The microdevice of claim 2, wherein said upper and lower surfaces are treated to modify the surface energy of said surfaces.

20. A microdevice comprising
a substrate comprising an upper surface, a lower surface and a plurality of channels in said substrate extending between said upper surface and said lower surface, each said channel being of a sufficiently small diameter to maintain a fluid inside said channel by capillary action, wherein either a portion of said upper and lower surfaces around said channel ends is recessed or each end of each of said channels in said substrate has a diameter sufficiently larger than that in the remaining portion of said channels away from said channel ends that any fluid substance in said channels is caused by capillary action to locate preferentially in said remaining portion of said channels.

21. A method for transferring a small quantity of a substance from an initial receptacle to a receiving receptacle, said method providing the steps of:
providing a microtransfer device, said device comprising a substrate, said substrate comprising an upper surface, a lower surface and a channel in said substrate extending between said upper surface and said lower surface, said channel being of a sufficiently small diameter to maintain a fluid inside said channel by capillary action;
providing an initial receptacle containing a fluid comprising said substance;
causing said fluid to wick into said channel by capillary action;
placing said device above a receiving receptacle, wherein said channel is in alignment with a receiving site in said receiving receptacle; and
transferring said substance from said channel to said receiving site.

22. The method of claim 21, wherein, following said causing step, said fluid comprising said substrate is dried in said channel.

23. The method of claim 21, wherein, in said providing step, said device comprises a plurality of channels.

24. The method of claim 23, wherein, in said providing step, either a portion of said upper and lower surfaces around said channel ends is recessed, or each end of each of said channels in said substrate has a diameter sufficiently larger than that in the remaining portion of said channels away from said channel ends that any fluid substance in said channels is caused by capillary action to locate preferentially in said remaining portion of said channels.

25. The method of claim 23, wherein, in said providing step, said device further comprises sealing material sealing said channel ends and said receiving receptacle comprises multiple receiving sites, said receiving sites containing a fluid, and wherein, following said placing step, said method further comprises the step of distorting said substrate to cause portions of said substrate comprising said channels to project away from portions of said substrate not comprising said channels, and said washing step comprises placing said portions of said substrate comprising said channels under the surface of said fluid in said receiving sites.

26. The method of claim 23, wherein multiple channels in said deviced are spaced in a group and said channels in said group are aligned with the same receiving site in said receiving receptacle.

27. The method of claim 26, wherein, in said causing step, the same sample substance is caused to wick into all channels in said group.

28. The method of claim 26, wherein, in said causing step, the different sample substances are caused to wick into different channels in said group.

29. A method for carrying out an assay, said method comprising the steps of:
providing a microdevice, said device comprising a substrate, said substrate comprising an upper surface, a lower surface and a channel in said substrate extending between said upper surface and said lower surface, said channel being of a sufficiently small diameter to maintain a fluid inside said channel by capillary action;
providing an initial receptacle containing a fluid comprising a sample substance;
causing said fluid to wick into said channel by capillary action;
placing said device above a receiving receptacle, wherein said channel is in alignment with a receiving site in said receiving receptacle;
either before or after said placing step, carrying out an assay on said sample substance; and
washing said sample substance from said channel to said receiving site.

30. The method of claim 29, said method further comprising the step of maintaining temperature control of said fluid in said filled device.

31. The method of claim 30, wherein said assay comprises cycling said temperature of said fluid.

32. The method of claim 31, wherein said assay is the polymerase chain reaction assay.

33. The method of claim 29, said method further comprising directly detecting the results of said assay.
